SEQUENCE LISTING (16 sequences)

SEQ ID NO: 1 - protein, 381 aa, Streptomyces sp.
MTQTTHHTPDTARQADPFPVKGMDAVVFAVGNAKQAAHYYSTAFGMQLVAYSGPENGSRETASYVLTNGSARFVLTSVIKPATPWGHFLADHVAEHGDGVVDLAIEVPDARAAHAYAIEHGARSVAEPYELKDEHGTVVLAAIATYGKTRHTLVDRTGYDGPYLPGYVAAAPIVEPPAHRTFQAIDHCVGNVELGRMNEWVGFYNKVMGFTNMKEFVGDDIATEYSALMSKVVADGTLKVKFPINEPALAKKKSQIDEYLEFYGGAGVQHIALNTGDIVETVRTMRAAGVQFLDTPDSYYDTLGEWVGDTRVPVDTLRELKILADRDEDGYLLQIFTKPVQDRPTVFFEIIERHGSMGFGKGNFKALFEAIEREQEKRGNL

SEQ ID NO: 2 - protein, 381 aa, synthetic construct
MTQTTHHTPDTARQADPFPVKGMDAVVFAVGNAKQAAHYYSTAFGMQLVAYSGPENGSRETASYVLTNGSARFVLTSVIKPATPWGHFLADHVAEHGDGVVDLAIEVPDARAAHAYAIEHGARSVAEPYELKDEHGTVVLAAIATYGKTRHTLVDRTGYDGPYLPGYVAAAPIVEPPAHRTFQAIDHCAGNVELGRMNEWVGFYNKVMGFTNMKEAVGDDIATEYSALMSKVVADGTLKVKFAIQEPALAKKKSAIDEYLEFYGGAGVQHIALNTGDIVETVRTMRAAGVQFLDTPDSYYDTLGEWVGDTRVPVDTLRELKILADRDEDGYLLQIFTKPVQDRPTVFFEIIERHGSMGFGKGNFKAIFEAIEREQEKRGNL

SEQ ID NO: 3 - protein, 381 aa, synthetic construct
MTQTTHHTPDTARQADPFPVKGMDAVVFAVGNAKQAAHYYSTAFGMQLVAYSGPENGSRETASYVLTNGSARFVLTSVIKPATPWGHFLADHVAEHGDGVVDLAIEVPDARAAHAYAIEHGARSVAEPYELKDEHGTVVLAAIATYGKTRHTLVDRTGYDGPYLPGYVAAAPIVEPPAHRTFQAIDHCAGAVELGRMNEWVGFYNKVMGFTNMKEFVGDDIATEYSALMLKVVADGTLKVKFGIFEPALAKKKSPIDEYLEFYGGAGVQHIALNTGDIVETVRTMRAAGVQFLDTPDSYYDTLGEWVGDTRVPVDTLRELKILADRDEDGYLLQIFTKPVQDRPTVFFEIIERHGSMGFGKGNFKAIFEAIEREQEKRGNL

SEQ ID NO: 4 - protein, 357 aa, Amycolatopsis orientalis
MQNFEIDYVEMYVENLEVAAFSWVDKYAFAVAGTSRSADHRSIALRQGQVTLVLTEPTSDRHPAAAYLQTHGDGVADIAMATSDVAAAYEAAVRAGAEAVRAPGQHSEAAVTTATIGGFGDVVHTLIQRDGTSAELPPGFTGSMDVTNHGKGDVDLLGIDHFAICLNAGDLGPTVEYYERALGFRQIFDEHIVVGAQAMNSTVVQSASGAVTLTLIEPDRNADPGQIDEFLKDHQGAGVQHIAFNSNDAVRAVKALSERGVEFLKTPGAYYDLLGERITLQTHSLDDLRATNVLADEDHGGQLFQIFTASTHPRHTIFFEVIERQGAGTFGSSNIKALYEAVELERTGQSEFGAARR

SEQ ID NO: 5 - protein, 381 aa, Streptomyces avermitilis; identical to SEQ ID NO: 1

SEQ ID NO: 6 - protein, 331 aa, unidentified
MGSVSKANVPKIDVSPLFGDDQAAKMRVAQQIDAASRDTGFFYAVNHGINVQRLSQKTKEFHMSITPEEKWDLAIRAYNKEHQDQVRAGYYLSIPGKKAVESFCYLNPNFTPDHPRIQAKTPTHEVNVWPDETKHPGFQDFAEQYYWDVFGLSSALLKGYALALGKEENFFARHFKPDDTLASVVLIRYPYLDPYPEAAIKTAADGTKLSFEWHEDVSLITVLYQSNVQNLQVETAAGYQDIEADDTGYLINCGSYMAHLTNNYYKAPIHRVKWVNAERQSLPFFVNLGYDSVIDPFDPREPNGKSDREPLSYGDYLQNGLVSLINKNGQT

SEQ ID NO: 7 - protein, 198 aa, Streptomyces viridochromogenes
MSNTKTASTGFAELLKDRREQVKMDHAALASLLGETPETVAAWENGEGGELTLTQLGRIAHVLGTSIGALTPPAGNDLDDGVIIQMPDERPILKGVRDNVDYYVYNCLVRTKRAPSLVPLVVDVLTDNPDDAKFNSGHAGNEFLFVLEGEIHMKWGDKENPKEALLPTGASMFVEEHVPHAFTAAKGTGSAKLIAVNF

SEQ ID NO: 8 - protein, 297 aa, Chromobacterium violaceum
MNDRADFVVPDITTRKNVGLSHDANDFTLPQPLDRYSAEDHATWATLYQRQCKLLPGRACDEFMEGLERLEVDADRVPDFNKLNQKLMAATGWKIVAVPGLIPDDVFFEHLANRRFPVTWWLREPHQLDYLQEPDVFHDLFGHVPLLINPVFADYLEAYGKGGVKAKALGALPMLARLYWYTVEFGLINTPAGMRIYGAGILSSKSESIYCLDSASPNRVGFDLMRIMNTRYRIDTFQKTYFVIDSFKQLFDATAPDFAPLYLQLADAQPWGAGDVAPDDLVLNAGDRQGWADTEDV

SEQ ID NO: 9 - protein, 446 aa, unidentified
MTGVAVPHRAELARQLIDARNRTLRLVDFDDAELRRQYDPLMSPLVWDLAHIGQQEELWLLRGGDPRRPGLLEPAVEQLYDAFVHPRASRVHLPLLSPAQARRFCATVRSAVLDALDRLPEDADTFAFGMVVSHEHQHDETMLQALNLRSGEPLLGSGTALPPGRPGVAGTSVLVPGGPFVLGVDLADEPYALDNERPAHVVDVPAFRIGRVPVTNAEWRAFIDDGGYRQRRWWSDAGWAYRCEAGLTAPQFWNPDGTRTRFGHVEDIPPDEPVQHVTYFEAEAYAAWAGARLPTEIEWEKACAWDPATGRRRRYPWGDAAPTAALANLGGDALRPAPVGAYPAGASACGAEQMLGDVWEWTSSPLRPWPGFTPMIYQRYSQPFFEGAGSGDYRVLRGGSWAVAADILRPSFRNWDHPIRRQIFAGVRLAWDVDRQTARPGPVGGC

SEQ ID NO: 10 - protein, 216 aa, Escherichia coli
MLDLFADAEPWQEPLAAGAVILRRFAFNAAEQLIRDINDVASQSPFRQMVTPGGYTMSVAMTNCGHLGWTTHRQGYLYSPIDPQTNKPWPAMPQSFHNLCQRAATAAGYPDFQPDACLINRYAPGAKLSLHQDKDEPDLRAPIVSVSLGLPAIFQFGGLKRNDPLKRLLLEHGDVVVWGGESRLFYHGIQPLKAGFHPLTIDCRYNLTFRQAGKKE

SEQ ID NO: 11 - protein, 310 aa, Pseudomonas syringae
MSKKFALTAEQRASFEKNGFIGPFDAYSPEEMKETWKRTRLRLLDRSAAAYQDLDAISGGTNIANYDRHLDDDFLASHICRPEICDRVESILGPNVLCWRTEFFPKYPGDEGTDWHQADTFANASGKPQIIWPENEEFGGTITVWTAFTDANIANGCLQFIPGTQNSMNYDETKRMTYEPDANNSVVKDGVRRGFFGYDYRQLQIDENWKPDEASAVPMQMKAGQFIIFWSTLMHASYPHSGESQEMRMGFASRYVPSFVHVYPDSDHIEEYGGRISLEKYGAVQVIGDETPEYNRLVTHTTRGKKFEAV

SEQ ID NO: 12 - protein, 240 aa, unidentified
MCAPLEKDDIRRLSQAFHRFGIVTVTELIEPHTRKLVRAEADRLLDQYAERRDLRLATTDYTRRSMSVVPSETIAANSELVTGLYAHRELLAPLEAIAGERLHPCPKADEEFLITRQEQRGDTHGWHWGDFSFALIWVLQAPPIDVGGLLQCVPHTTWDKASPQINRYLVENPIDTYHFESGDVYFLRTDTTLHRTIPLREDTTRIILNMTWAGERDLSRKLAADDRWWDNAEVSAARAI

SEQ ID NO: 13 - protein, 273 aa, Burkholderia ambifaria
MQHTYPAQLMRFGTAARAEHMTIAAAIHALDADEADAIVMDIVPDGERDAWWDDEGFSSSPFTKNAHHAGIVATSVTLGQLQREQGDKLVSKAAEYFGIACRVNDGLRTTRFVRLFSDALDAKPLTIGHDYEVEFLLATRRVYEPFEAPFNFAPHCDDVSYGRDTVNWPLKRSFPRQLGGFLTIQGADNDAGMVMWDNRPESRAALDEMHAEYRETGAIAALERAAKIMLKPQPGQLTLFQSKNLHAIERCTSTRRTMGLFLIHTEDGWRMFD

SEQ ID NO: 14 - protein, 252 aa, Micromonospora carbonacea
MDAMEVVGTIDHRDREEFRSRGFAILPQVASESEVAWLRQAYDRLFVRRATPGAEDFYDIAGQRDREGPPLLPQIIKPEKYVPELLDSPHFARCRSIASAFLDMAEEELEFYGHAILKPPRYGAPTPWHQDEAYMDPRWRRRGLSIWTTLDEATVESGCLHYLPGGHRGPVLPHHHIDNDDRIRGLMTDDVDPTSAVACPLAPGGAVVHDFRTPHYAGPNLTDQPRRAYVLVFMSAPAEVADPEPRPWMDWG

SEQ ID NO: 15 - protein, 280 aa, Mesorhizobium sp.
MTTRILGVVQLDQRRLTDDLAVLAKSNFSSEYSDFACGRWEFCMLRNQSGKQEEQRVVVHETPALATPLGQSLPYLNELLDNHFDRDSIRYARIIRISENACIIPHRDYLELEGKFIRVHLVLDTNEKCSNTEENNIFHMGRGEIWFLDASLPHSAGCFSPTPRLHLVVDIEGTRSLEEVAINVEQPSARNATVDTRKEWTDETLESVLGFSEIISEANYREIVAILAKLHFFHKVHCVDMYGWLKEICRRRGEPALIEKANSLERFYLIDRAAGEVMTY

SEQ ID NO: 16 - protein, 243 aa, Streptomyces sp.
MLTRPTAALSSPADITGDLVRTGFSMVPGSDMRVPAALQDSLKTLAASYDDLPADPYLPDGGNYRYRRHTRYTWRPATGELLVADNPGYFQTVENNAFAGGQWRKYEELTDEVREGAFLTALIDFNVGRLPLPEVEQWAVQVHCVRIVARDDAQGRPTPEGVHRDGCTYVSLHMVNRHNISGGRTSVYTPEHELITEKVFTDCLDSFFGDDPRVRHGVADVSVADPSLGEGTRDMLLMSYDPM

(19) United States
(12) Patent Application Publication
(10) Pub. No.: US 2025/0263677 A1
(43) Pub. Date: Aug. 21, 2025

(54) BIOCATALYTIC USE OF NONHEME IRON PROTEINS FOR MOLECULAR FUNCTIONALIZATION

(71) Applicant: The Johns Hopkins University, Baltimore, MD (US)

(72) Inventors: Xiongyi Huang, Baltimore, MD (US); Anthony J. Huls, Baltimore, MD (US); Qun Zhao, Baltimore, MD (US); Jinyan Rui, Baltimore, MD (US); Zhenhong Chen, Baltimore, MD (US); James Zhang, Baltimore, MD (US)

(21) Appl. No.: 18/859,481

(22) PCT Filed: May 16, 2023
(86) PCT No.: PCT/US2023/022431

§ 371 Date: Oct. 23, 2024

Related U.S. Application Data
(60) Provisional application No. 63/343,062, filed on May 17, 2022.

(51) Int. Cl. C12N 9/02; C12P 13/00

(52) U.S. Cl. CPC C12N 9/0069; C12P 13/00; C12Y 113/11027

(57) ABSTRACT

Provided herein are methods of functionalizing C(sp3)-H bonds using reprogramed metalloenzymes to perform radical-relay C(sp3)-H functionalization, activating a (sp3)-H bond via a reactive radical (X·) via hydrogen atom transfer (HAT); intercepting of the resulting carbon-centered radical by a redox-reactive metal complex; and obtaining a functionalized C—Y bond.

### CROSS REFERENCE TO RELATED APPLICATIONS

The present application claims the benefit under U.S.C. § 119(e) to U.S. Provisional Application Ser. No. 63/343,062, filed on May 17, 2022, the entire contents of which is incorporated herein by reference in its entirety.

### STATEMENT OF GOVERNMENT SUPPORT

This invention was made with government support under grant GM129419 awarded by the National Institutes of Health. The government has certain rights in the invention.

### INCORPORATION OF SEQUENCE LISTING

The instant application contains a Sequence Listing which has been submitted electronically in XML file format and is hereby incorporated by reference in its entirety. Said XNL copy, created on May 16, 2023, is named JHU4520-1_SL.xml and is 19,561 bytes in size.

### BACKGROUND OF THE INVENTION

### Field of the Invention

This invention relates generally to biochemical machinery for activating C—H bonds and more specifically to using reprogramed metalloenzymes to perform radical-relay functionalization to obtain C—N, C—S, C—C, and/or C-Halogen bonds.

### Background Information

The past decades have witnessed burgeoning advancement of biocatalytic methods for molecular functionalization. Capitalizing on the genetic tunability, broad functional group tolerance, and exquisite selectivity of protein catalysts, biocatalysis has found wide applications in drug development by providing access to medicinally valuable compounds that are challenging to make via classical chemical methods. In this regard, one important type of biosynthetic transformations that can greatly benefit drug discovery is enzymatic C—H functionalization. Such methods offer efficient means of diversifying drug leads and can significantly accelerate pharmaceutical discovery process by directly converting ubiquitous C—H bonds in molecules into functional groups of medicinal interest. While holding considerable potential in biomedical applications, enzymatic C—H functionalization only encompasses a limited set of transformations that have been acquired via natural evolution.

Enzymes that functionalize C(sp³)-H bonds are essential in a variety of biological processes ranging from xenobiotic metabolism to post translational modification of proteins. To support these diverse functions, nature has evolved a multitude of reactive intermediates to activate C(sp³)-H bonds, including 5′-deoxyadenosyl radical, glycyl radical, flavin-hydroperoxide, and high-valent metal-oxo, (hydro)peroxo, hydroxo, and superoxo complexes. While enabling a broad spectrum of biotransformations, these reactive species can only access a limited set of C(sp³)-H functionalization reactions. Many C(sp³)-H activation modes widely exploited in organic synthesis are noticeably absent in the current catalytic repertoire of biology, which constrains the scope and synthetic applications of C(sp³)-H functionalizing enzymes. A promising strategy to expand the scope of enzymatic C(sp³)-H functionalization is to engineer natural proteins to enable abiological reaction mechanisms for C(sp³)-H activation. This approach would combine the genetic tunability of natural proteins with the diversity of non-natural reaction mechanisms for C(sp³)-H activation. Thus far, research efforts in this field have been mostly focused on reactions mediated by metal-carbene and metal-nitrene intermediates. Despite this progress, the activation modes for enzymatic C(sp³)-H functionalization are still narrower than those of synthetic catalysis.

The installation of many medicinally important functional groups such as fluorine, trifluoromethyl, and nitrile groups are currently beyond the reach of catalytic capabilities of enzymatic C—H functionalization. This limitation continues to constrain the range of molecular structures that can be accessed via enzymatic catalysis. A promising strategy to expand the scope of enzymatic catalysis is to repurpose existing proteins to catalyze non-natural synthetic reactions. Accordingly, new enzymatic reactions for the installation of biomedically important chemical functionalities into organic molecules are needed.

### SUMMARY OF THE INVENTION

Provided herein are enzymatic systems that can operate radical relay reaction mechanism for C—H functionalization reactions.

In one aspect, the present invention provides a non-heme metalloenzyme with at least about 70% sequence identity to SEQ ID NO:1 and at least 1 mutation relative to SEQ ID NO:1. In some embodiments, the non-heme metalloenzyme has at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO:1. In some embodiments, the at least 1 mutation includes at least 2, at least 3, at least 4, at least 5, at least 6, at least 7, at least 8, at least 9, at least 10, at least 11, at least 12, at least 13, at least 14, or at least 15 mutations relative to SEQ ID NO:1. In some embodiments, the at least 1 mutation is at SEQ ID NO:1 position H187, V189, N191, L228, S230, P243, N245, Q255, Q269, H270, F336, E349, F364, L367, F368, or a combination thereof. In some embodiments, the at least 1 mutation is at least 2, at least 3, at least 4, at least 5, at least 6, at least 7, at least 8, at least 9, at least 10, at least 11, at least 12, at least 13, at least 14, or at least 15 mutations at SEQ ID NO:1 positions selected from H187, V189, N191, L228, S230, P243, N245, Q255, Q269, H270, F336, E349, F364, L367, and F368. In some embodiments, the at least 1 mutation is selected from V189A, N191A, F216A, S230L, P243A, P243G, N245Q, N245F, Q255A, Q255P, and L367I. In some embodiments, the at least 1 mutation diminishes active site volume in the non-heme metalloenzyme.

In another aspect, the present invention provides a non-heme metalloenzyme has at least at least 98.2%, at least 98.4%, at least 98.6%, at least 98.8%, at least 99%, at least 99.2%, at least 99.4%, or at least 99.6% sequence identity to SEQ ID NO:2 or SEQ ID NO:3.

In some embodiments, the non-heme metalloenzyme has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to any one of SEQ ID NO:1-16. In some embodiments, the non-heme metalloenzyme has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO:1. In some embodiments, the non-heme metalloenzyme has at least one, at least two, at least three, at least four, at least five, at least six, at least seven, at least eight, at least nine, at least ten, at least eleven, at least twelve, at least thirteen, at least fourteen, or fifteen mutations relative to SEQ ID NO:1 at positions selected from H187, V189, N191, L228, S230, P243, N245, Q255, Q269, H270, F336, E349, F364, L367, and F368.

In a further aspect, the present invention provides a composition that includes a non-heme metalloenzyme, an organic substrate with a C—H bond, and one or more of a halogen source, a nucleophile source, and a radical precursor.

In certain aspects, the present invention provides a method for modifying an organic substrate by: contacting the organic substrate with a non-heme metalloenzyme; abstracting a hydrogen atom from the organic substrate; and coupling a nucleophile to the organic substrate, thereby converting the organic substrate to a modified organic substrate. In some embodiments, the nucleophile is coupled to the carbon atom from which the hydrogen atom is abstracted. In some embodiments, the nucleophile is an azide, a halogen, a nitrile, a thiocyanate, a nitro, a cyanide, an alkoxide, a thiolate, an amine, a sulfonamide, an amide, a heteroaryl, or a trifluoromethyl. In some embodiments, the nucleophile is an azide or a halogen. In some embodiments, the nucleophile is an azide. In some embodiments, the nucleophile is a halogen. In some embodiments, the method has a chemoselectivity for azidation over fluorination of greater than about 3:2, greater than about 2:1, greater than about 3:1, greater than about 4:1, greater than about 5:1, greater than about 6:1, greater than about 7:1, greater than about 8:1, greater than about 9:1, greater than about 10:1, greater than about 12:1, greater than about 15:1, greater than about 20:1, or greater than about 25:1.

In some embodiments, the non-heme metalloenzyme catalyzes the coupling between the nucleophile and the organic substrate. In some embodiments, the nucleophile is bonded to a metal cofactor of the non-heme metalloenzyme prior to the coupling. In some embodiments, the hydrogen atom is abstracted from a carbon atom of the organic substrate. In some embodiments, the nucleophile is coupled to the carbon atom from which the hydrogen atom is abstracted. In some embodiments, the non-heme metalloenzyme has an iron cofactor, a copper cofactor, a cobalt cofactor, a manganese cofactor, a nickel cofactor, or a chromium cofactor. In particular embodiments, the non-heme metalloenzyme has an iron cofactor. In some embodiments, the iron cofactor has a +2 oxidation state. In some embodiments, the iron cofactor interconverts between +2 and +3 oxidation states. In some embodiments, the iron cofactor does not adopt a +4 oxidation state.

In some embodiments, the nucleophile is derived from a nucleophile source with a structure according to any one of Formulas (VIII)-(XVII) or (XIX):

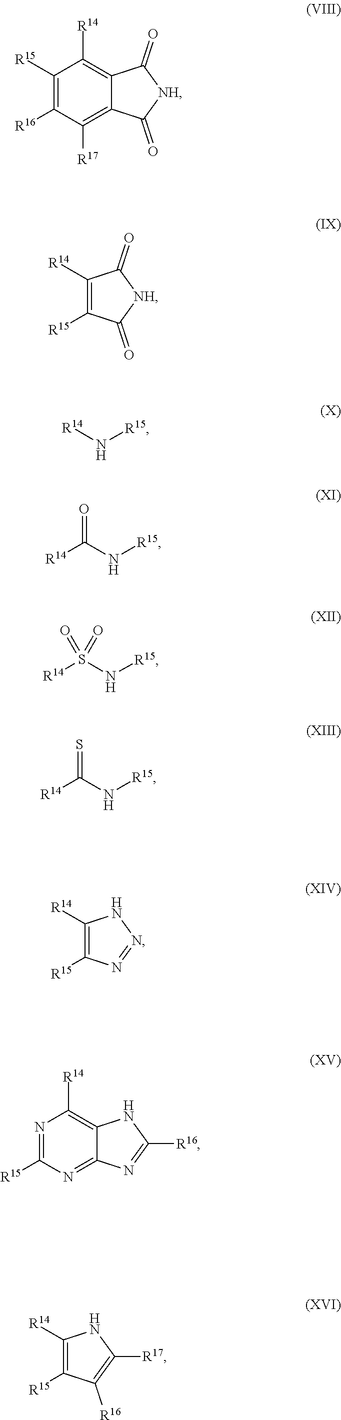

or M⁺X⁻ (XIX); wherein each instance of R¹⁴, R¹⁵, R¹⁶ and R¹⁷ is independently —H, optionally substituted C₁₋₁₈ alkyl, C₁₋₁₈ polyfluoroalkyl, optionally substituted C₂₋₁₈ alkenyl, optionally substituted C₂₋₁₈ alkynyl, optionally substituted C₆₋₁₀ aryl, optionally substituted 6- to 10-membered heteroaryl, optionally substituted 6- to 10-membered heterocyclyl, cyano, halo, nitro, —NR¹⁸R¹⁹, —BR²¹R²², —SiR¹⁸R¹⁹R²⁰, —C(O)OR¹⁸, —C(O)SR¹⁸, —C(O)NR¹⁸R¹⁹, —C(O)R¹⁸, —C(O)ONR¹⁸R¹⁹, —C(O)NR¹⁸OR¹⁹, —C(O)C(O)OR¹⁸, —S(O)OR¹⁸, —S(O)SR¹⁸, —S(O)NR¹⁸R¹⁹, —S(O)R¹⁸, —S(O)ONR¹⁸R¹⁹, —S(O)NR¹⁸OR¹⁹, —S(O)C(O)OR¹⁸, —S(O)₂OR¹⁸, —S(O)₂SR¹⁸, —S(O)₂NR¹⁸R¹⁹, —S(O)₂R¹⁸, —S(O)₂ONR¹⁸R¹⁹, —S(O)₂NR¹⁸OR¹⁹, —S(O)₂C(O)OR¹⁸, or —P(O)(OR¹⁸)(OR¹⁹); each instance of R¹⁸, R¹⁹, and R²⁰ is independently —H, C₁-C₃ alkyl, or C₁-C₃ haloalkyl; each instance of R²¹ and R²² is independently —H, C₁-C₃ alkyl, C₁-C₃ haloalkyl, or —OR¹⁸; M⁺ is Na⁺, K⁺, Cs⁺, or [N(R¹²)₄]⁺; X⁻ is F⁻, Cl⁻, Br⁻, I⁻, N₃⁻, SCN⁻, CN⁻, NCO⁻, [SR¹³]⁻, or [OR¹³]⁻; each instance of R¹² is independently —H, C₁-C₆ alkyl, or C₁-C₆ haloalkyl, or wherein two instances of R¹² are taken together along with the nitrogen to which they are attached to form a C₂-C₈ heterocycloalkyl; and each instance of R¹³ is independently —H, C₁-C₆ alkyl, or C₁-C₆ haloalkyl.

In one embodiment, the hydrogen atom is abstracted by an organic radical generated by the non-heme metalloenzyme. In a particular embodiment, the organic radical is generated through homolysis of a bond on a radical precursor. In some embodiments, the radical precursor is coupled to the organic substrate. In some embodiments, the bond on the radical precursor is a halogen-halogen bond, a carbon-halogen bond, a nitrogen-halogen bond, or an oxygen-oxygen bond. In some embodiments, the radical precursor has a structure according to any one of Formulas (I)-(VII):

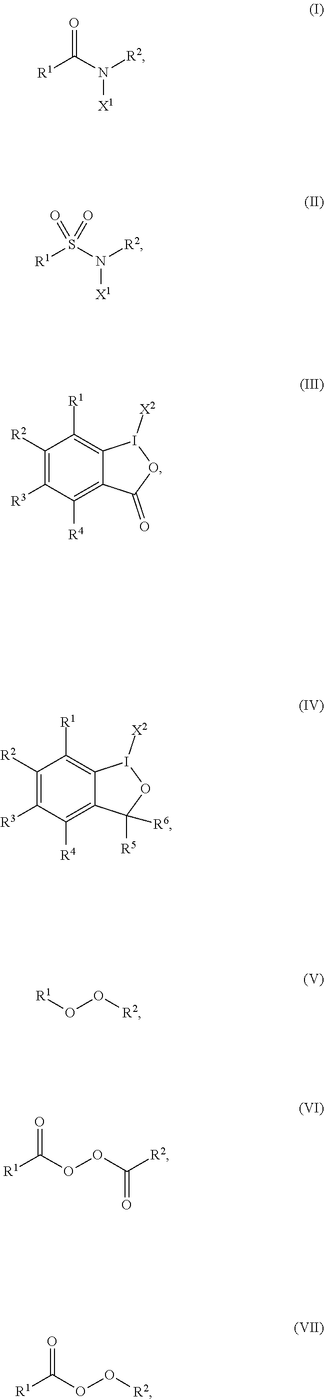

wherein each instance of R¹, R², R³, R⁴, R⁵, and R⁶ is independently the organic substrate, —H, optionally substituted C₁₋₁₈ alkyl, optionally substituted C₁₋₁₈ polyfluoroalkyl, optionally substituted C₂₋₁₈ alkenyl, optionally substituted C₂₋₁₈ alkynyl, optionally substituted C₆₋₁₀ aryl, optionally substituted 6- to 10-membered heteroaryl, optionally substituted 6- to 10-membered heterocyclyl, cyano, halo, nitro, —NR⁷R⁸, —BR¹⁰R¹¹, —SiR⁷R⁸R⁹, —C(O)OR⁷, —C(O)SR⁷, —C(O)NR⁷R⁸, —C(O)R⁷, —C(O)ONR⁷R⁸, —C(O)NR⁷OR⁸, —C(O)C(O)OR⁷, —S(O)OR⁷, —S(O)SR⁷, —S(O)NR⁷R⁸, —S(O)R⁷, —S(O)ONR⁷R⁸, —S(O)NR⁷OR⁸, —S(O)C(O)OR⁷, —S(O)₂OR⁷, —S(O)₂SR⁷, —S(O)₂NR⁷R⁸, —S(O)₂R⁷, —S(O)₂ONR⁷R⁸, —S(O)₂NR⁷OR⁸, —S(O)₂C(O)OR⁷, or —P(O)(OR⁷)(OR⁸); each instance of R⁷, R⁸, and R⁹ is independently —H, C₁-C₃ alkyl, or C₁-C₃ haloalkyl; each instance of R¹⁰ and R¹¹ is independently —H, C₁-C₃ alkyl, C₁-C₃ haloalkyl, or —OR⁷; each instance of X¹ is independently —F, —Cl, —Br, or —I; and each instance of X² is independently —F, —Cl, or —Br.

In some embodiments, the modified organic substrate is coupled to the nucleophile through a carbon-nitrogen bond, a carbon-sulfur bond, a carbon-carbon bond, or a carbon halogen bond. In some embodiments, the organic substrate contains a carbon-halogen or nitrogen-halogen bond that is not cleaved during the method. In particular embodiments, the method further includes dehalogenating the organic substrate. In further embodiments, the method is performed under anaerobic conditions. In some embodiments, the modified organic substrate has an enantiomeric ratio of at least about 60:40, at least about 65:35, at least about 70:30, at least about 75:25, at least about 80:20, at least about 85:15, at least about 90:10, or at least about 95:5. In some embodiments, the non-heme metalloenzyme has a total turnover of at least about 100, at least about 200, at least about 300, at least about 400, at least about 500, at least about 600, at least about 700, at least about 800, at least about 900, at least about 1000, at least about 1200, at least about 1500, at least about 2000, at least about 2500, at least about 3000, at least about 4000, at least about 5000, at least about 6000, at least about 8000, or at least about 10000. In some embodiments, method is performed in the presence of a cell that expresses the non-heme metalloenzyme.

In some embodiments, the organic substrate has a structure according to Formula (XVIII):

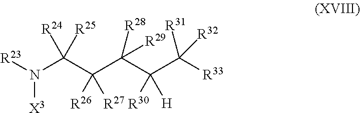

wherein R²³, R²⁴, R²⁵, R²⁶, R²⁷, R²⁸, R²⁹, R³⁰, R³¹, R³², and R³³ are independently —H, optionally substituted C₁₋₁₈ alkyl, C₁₋₁₈ polyfluoroalkyl, optionally substituted C₂₋₁₈ alkenyl, optionally substituted C₂₋₁₈ alkynyl, optionally substituted C₆₋₁₀ aryl, optionally substituted 6- to 10-membered heteroaryl, optionally substituted 6- to 10-membered heterocyclyl, cyano, halo, nitro, —NR³⁴R³⁵, —BR³⁷R³⁸, —SiR³⁴R³⁵R³⁶, —C(O)OR³⁴, —C(O)SR³⁴, —C(O)NR³⁴R³⁵, —C(O)R³⁴, —C(O)ONR³⁴R³⁵, —C(O)NR³⁴OR³⁵, —C(O)C(O)OR³⁴, —S(O)OR³⁴, —S(O)SR³⁴, —S(O)NR³⁴R³⁵, —S(O)R³⁴, —S(O)ONR³⁴R³⁵, —S(O)NR³⁴OR³⁵, —S(O)C(O)OR³⁴, —S(O)₂OR³⁴, —S(O)₂SR³⁴, —S(O)₂NR³⁴R³⁵, S(O)₂R³⁴, S(O)₂ONR³⁴R³⁵, —S(O)₂NR³⁴OR³⁵, —S(O)₂C(O)OR³⁴, or —P(O)(OR³⁴)(OR³⁵); each instance of R³⁴, R³⁵, and R³⁶ is independently —H, C₁-C₃ alkyl, or C₁-C₃ haloalkyl; each instance of R³⁷ and R³⁸ is independently —H, C₁-C₃ alkyl, C₁-C₃ haloalkyl, or —OR³⁴; and X₃ is —F, —Cl, —Br, or —I. In some embodiments, X³ is abstracted by the non-heme metalloenzyme.

An additional aspect of the present invention provides a method of functionalizing C(sp³)-H bonds by: using reprogramed metalloenzymes to perform radical-relay C(sp³)-H functionalization; activating a (sp³)-H bond via a reactive radical (X·) via hydrogen atom transfer (HAT); intercepting of the resulting carbon-centered radical by a redox-reactive metal complex; and obtaining a functionalized C—Y bond, thereby functionalizing C(sp³)-H bonds. In some embodiments, the reprogrammed metalloenzymes are non-heme iron enzymes. In some embodiments, the reprogrammed metalloenzymes are enantioselective variants. In some embodiments, the reactive radical (X·) is a nitrogen radical (N·) and/or an oxygen radical (O·). In some embodiments, the functionalized C—Y bond is a C—C, C—S, C—N, C—F, and/or, C-halogen bond.

In one example, a series of enzymes are engineered for building C—C, C—S, C—N, C—F, and C-halogen bonds via C—H bond functionalization via a nitrogen- or oxygen-centered radicals.

Also provided are collections of non-heme enzyme-based biocatalysts that can directly functionalize inert C(sp³)-H bonds to install biomedically relevant chemical moieties such as azide, chlorine, nitrile, thiocyanate, nitro, and trifluoromethyl groups.

### BRIEF DESCRIPTION OF THE DRAWINGS

FIG. 1 illustrates the mechanism of enzymatic system that can operate radical relay reaction mechanism for C—H functionalization reactions.

FIG. 2 illustrates mechanism and optimized conditionals for fluorination.

FIGS. 3A-3C is an illustration with reaction products generated from exemplary substrate compounds. FIG. 3A shows the scope of Sav HppD Az1 and Sav HppD Az2-transformed products. Experiments were performed at analytical scale using suspensions of E. coli expressing Sav HppD variants in KPi buffer (pH 7.4) at room temperature under anaerobic conditions for 24 hours. The absolute configuration of enzymatically synthesized azidation product 1N was determined to be S via X-ray crystallography. The absolute configurations of all other azidation products were inferred by analogy. FIG. 3B summarizes a preparative scale synthesis and absolute configuration determination of product 1N. FIG. 3C is a reaction scheme for a one-pot chemoenzymatic synthesis of azidation product 11N followed by copper catalyzed azide-alkyne cycloaddition.

FIG. 4 is an illustration of O-radical directed functionalization and results of such reactions.

FIG. 5 is a thiocyanation reaction scheme and NMR data showing the progress of a thiocyanation reaction.

FIG. 6 is an intermolecular radical relay azidation mechanism and a table showing activity screening data with various metalloenzymes.

FIG. 7 is a set of tables with enantioselectivity data.

FIGS. 8A-8C is a set of reaction schemes that cover enzymatic and non-enzymatic radical relay mechanisms. FIG. 8A is a reaction scheme for a radical relay C—H functionalization that involves an initial hydrogen atom transfer (HAT) mediated by a heteroatom-centered radical (X·) followed by the trapping of the carbon-centered radical with redox-active metal complex. FIG. 8B is a reaction scheme for a mechanism employed by natural non-heme iron enzymes for C(sp³)-H halogenation/azidation. FIG. 8C is a reaction scheme for a mechanism which integrates radical relay chemistry into non-heme iron enzymes to enable unnatural C—H functionalization reactions.

FIGS. 9A-9C is a computational model of the Sav HppD active site, a reaction scheme, and a plot of total turnovers for various Sav HppD variants. FIG. 9A shows protein residues selected for mutagenesis from among: (1) loop residues surrounded the active site (N191, F216, Q255, F359), (2) residues on the C-terminal a-helix (K361, L367, N363), and (3) residues on the β barrel of the C-terminal domain (V189, S230, P243, N245, Q269, Q334, F336, R353). The computational model was generated from protein database entry 1T47. FIG. 9B provides the azidation reaction scheme for the reaction screened with a high-throughput screening platform. FIG. 9C provides representative variants identified during the directed evolution of Sav HppD. Experiments were performed at analytical scale using suspensions of E. coli expressing Sav HppD variants (OD₆₀₀=10), 10 mM substrate 1NF, 25 mM NaN₃, 2.5 mM Fe²⁺ in KPi buffer (pH 7.4) at room temperature under anaerobic conditions for 24 hours.

FIG. 10 is a series of plots which summarize kinetics for non-heme iron enzyme mediated azidation reactions. The leftmost plot provides kinetics data for wild-type Sav HppD. The middle plot provides kinetics data for Az1 Sav HppD. The rightmost plot provides kinetics data for Az2 Sav HppD.

FIG. 11 is an azidation reaction scheme and a table listing enantioselectivities for an azidation reaction mediated by several non-heme metalloenzymes.

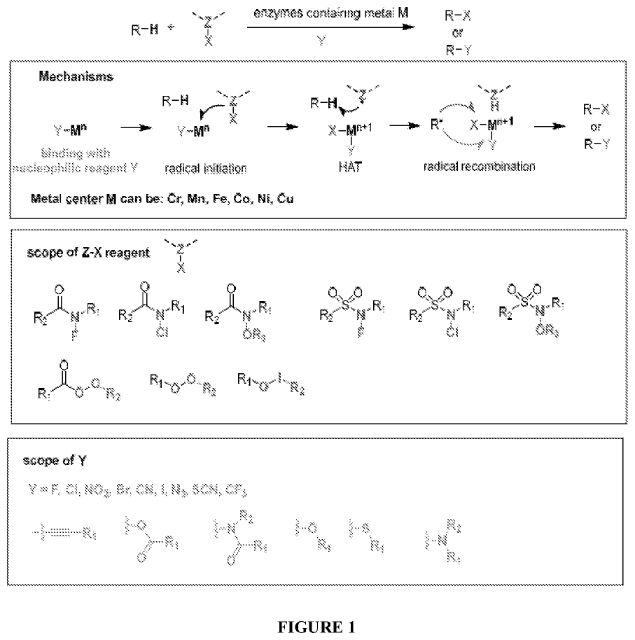

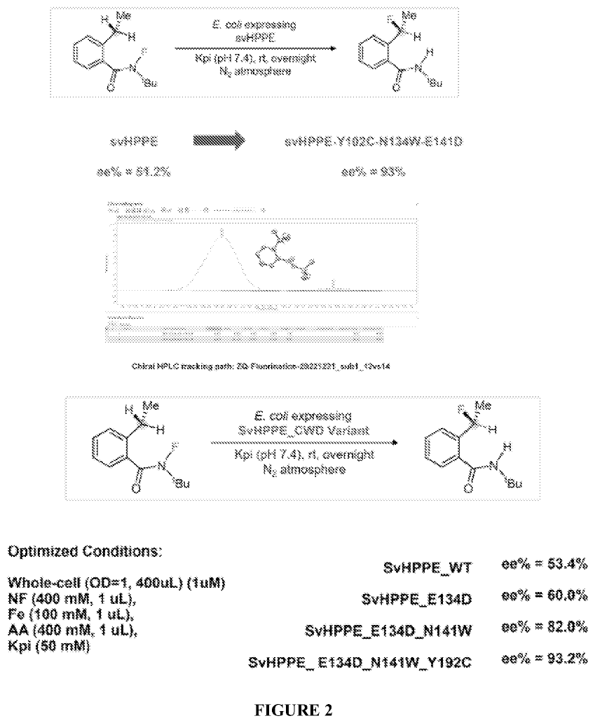

### DETAILED DESCRIPTION OF THE INVENTION

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., CH₂O is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like. Further, more generally, a “carbyl” refers to a carbon atom or a moiety comprising one or more carbon atoms acting as a bivalent radical.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom. Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

The term “haloalkyl,” by itself or in combination with another term, means, unless otherwise stated, a stable straight or branched chain, or cyclic hydrocarbon group, or combinations thereof, consisting of at least one carbon atoms and at least one halogen selected from the group consisting of F, Cl, Br, and I. Representative haloalkyl groups include —CH₂F, —CHClCH₃, —CHClCH₂Cl, —CH₂CH₂CF₂CF₃, and —CF(CF₂CF₃)₂.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The terms “heterocycloalkyl” and “cycloheteroalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl,” by themselves or in combination with other terms, represent, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₁₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), I-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbols [symbol] and - (e.g., as in —OH) denote the point of attachment of a moiety to the remainder of a molecule.

When a named atom of an aromatic ring or a heterocyclic aromatic ring is defined as being “absent,” the named atom is replaced by a direct bond.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms “halo,” “halide,” or “halogen” as used herein refer to fluoro, chloro, bromo, and iodo groups. Additionally, terms, such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an OH group.

The terms “azide” and “azido” refer to the group —N₃.

The term “peroxo” denotes an —O—OR′ end group or an —O—O— linking group.

The term polyfluoroalkyl refers to an alkyl group in which all hydrogens are replaced by fluoride. Examples of polyfluoroalkyl groups include —CF₃, —CF(CF₃)₂, and —CF₂CF₂CF₃.

The term “thiocyanate” as used herein refers to —S—C≡N group.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure. The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g., (+)-tartrates, (−)-tartrates or mixtures thereof including racemic mixtures, succinates, benzoates and salts with amino acids, such as glutamic acid. These salts may be prepared by methods known to those skilled in art. Also included are base addition salts, such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids, such as arginate and the like, and salts of organic acids like glucuronic or galactunoric acids and the like. Certain specific compounds of the present disclosure contain both basic and acidic functionalities that allow the compounds to be converted into either base or acid addition salts.

Disclosed herein are metalloenzyme-mediated methods for C—H bond activation. The methods can achieve H-atom abstraction (HAT) and form carbon-nitrogen, carbon-sulfur, carbon-carbon, and carbon-halogen bonds in a wide variety of substrates. The methods can be performed in vivo and in vitro, and are thus amenable to a range of bioorthogonal and synthetic applications.

As used herein, the term “H-atom abstraction” (HAT) denotes the removal of a hydrogen atom from a substrate. Formally, H-atom abstraction includes hydrogen bond homolysis, resulting in the removal of a proton or deuteron and an electron from the substrate. H-atom abstraction often generates an organic radical at the site of hydrogen atom removal on the substrate.

In certain aspects, the present invention provides a method for modifying an organic substrate by contacting the organic substrate with a non-heme metalloenzyme; abstracting a hydrogen atom from the organic substrate; and coupling a nucleophile to the organic substrate, thereby converting the organic substrate to a modified organic substrate. In some embodiments, the nucleophile is coupled to the carbon atom from which the hydrogen atom is abstracted. In some embodiments, the nucleophile is an azide, a halogen, a nitrile, a thiocyanate, a nitro, a cyanide, an alkoxide, a thiolate, an amine, a sulfonamide, an amide, a heteroaryl, or a trifluoromethyl. In some embodiments, the nucleophile is an azide or a halogen. In some embodiments, the nucleophile is an azide. In some embodiments, the nucleophile is a halogen. In some embodiments, the method has a chemoselectivity for azidation over fluorination of greater than about 3:2, greater than about 2:1, greater than about 3:1, greater than about 4:1, greater than about 5:1, greater than about 6:1, greater than about 7:1, greater than about 8:1, greater than about 9:1, greater than about 10:1, greater than about 12:1, greater than about 15:1, greater than about 20:1, or greater than about 25:1.

In some embodiments, the non-heme metalloenzyme catalyzes the coupling between the nucleophile and the organic substrate. In some embodiments, the nucleophile is bonded to a metal cofactor of the non-heme metalloenzyme prior to the coupling. In particular embodiments, the nucleophile is bonded to the metal cofactor of the non-heme iron enzyme prior to the hydrogen atom abstraction. For example, the metal cofactor can be bonded to an azide or halide that is transferred from the metal cofactor to the substrate following hydrogen atom abstraction from the substrate.

In particular aspects, the method includes contacting the organic substrate with a halogen source and a non-heme metalloenzyme, thereby abstracting a hydrogen from the organic substrate and coupling a halogen derived from the halogen source to the organic substrate. In some embodiments, the halogen is —F, —Cl, —Br, or —I. In some embodiments, the halogen is —F. A general outline for this reaction is provided in SCHEME 1.

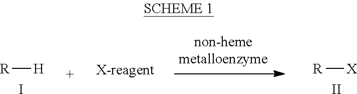

In some embodiments, the C—H bond is an allylic C—H bond, a benzylic C—H bond, a propargylic C—H bond, or an aliphatic C—H bond. In some embodiments, the C—H bond is an aliphatic C—H bond. In some cases, the organic substrate is coupled to the halogen source, such that the reaction is an intramolecular reaction.

In some embodiments, the halogen source has a structure according to any one of Formulas (I)—(IV):

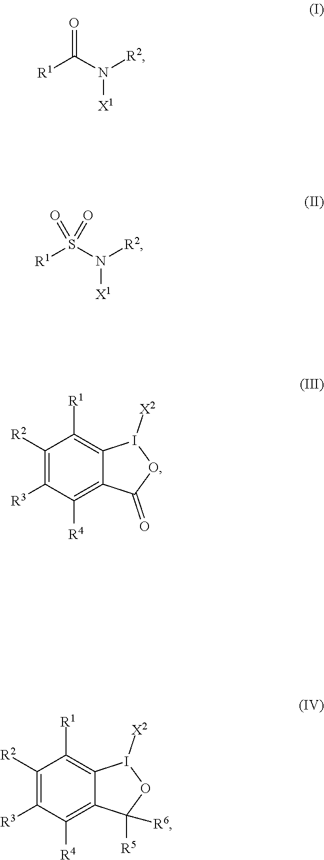

wherein:
each instance of R¹, R², R³, R⁴, R⁵, and R⁶ is independently the organic substrate, —H, optionally substituted C₁₋₁₈ alkyl, optionally substituted C₁₋₁₈ polyfluoroalkyl, optionally substituted C₂₋₁₈ alkenyl, optionally substituted C₂₋₁₈ alkynyl, optionally substituted C₆₋₁₀ aryl, optionally substituted 6- to 10-membered heteroaryl, optionally substituted 6- to 10-membered heterocyclyl, cyano, halo, nitro, —NR⁷R⁸, —BR¹⁰R¹¹, —SiR⁷R⁸R⁹, —C(O)OR⁷, —C(O)SR⁷, —C(O)NR⁷R⁸, —C(O)R⁷, —C(O)ONR⁷R⁸, —C(O)NR⁷OR⁸, —C(O)C(O)OR⁷, —S(O)OR⁷, —S(O)SR⁷, —S(O)NR⁷R⁸, —S(O)R⁷, —S(O)ONR⁷R⁸, —S(O)NR⁷OR⁸, —S(O)C(O)OR⁷, —S(O)₂OR⁷, —S(O)₂SR⁷, —S(O)₂NR⁷R⁸, —S(O)₂R⁷, —S(O)₂ONR⁷R⁸, —S(O)₂NR⁷OR⁸, —S(O)₂C(O)OR⁷, or —P(O)(OR⁷)(OR⁸);
each instance of R⁷, R⁸, and R⁹ is independently —H, C₁-C₃ alkyl, or C₁-C₃ haloalkyl;
each instance of R¹⁰ and R¹¹ is independently —H, C₁-C₃ alkyl, C₁-C₃ haloalkyl, or —OR⁷;
each instance of X¹ is independently —F, —Cl, —Br, or —I; and
each instance of X² is independently —F, —Cl, or —Br.

In some embodiments, each instance of X¹ is independently —F or —Cl. In some embodiments, each instance of X¹ is —F. In some embodiments, each instance of X² is independently —F or —Cl. In some embodiments, each instance of X² is —F.

In some embodiments, each instance of R¹, R², R³, R⁴, R⁵, and R⁶ is independently —H, optionally substituted C₁₋₁₈ alkyl, optionally substituted C₁₋₁₈ polyfluoroalkyl, optionally substituted C₂₋₁₈ alkenyl, optionally substituted C₂₋₁₈ alkynyl, optionally substituted C₆₋₁₀ aryl, optionally substituted 6- to 10-membered heteroaryl, optionally substituted 6- to 10-membered heterocyclyl, cyano, halo, nitro, —NR⁷R⁸, —BR¹⁰R¹¹, —SiR⁷R⁸R⁹, —C(O)OR⁷, —C(O)SR⁷, —C(O)NR⁷R⁸, —C(O)R⁷, —C(O)ONR⁷R⁸, —C(O)NR⁷OR⁸, —C(O)C(O)OR⁷, —S(O)OR⁷, —S(O)SR⁷, —S(O)NR⁷R⁸, —S(O)R⁷, —S(O)ONR⁷R⁸, —S(O)NR⁷OR⁸, —S(O)C(O)OR⁷, —S(O)₂OR⁷, —S(O)₂SR⁷, —S(O)₂NR⁷R⁸, —S(O)₂R⁷, —S(O)₂ONR⁷R⁸, —S(O)₂NR⁷OR⁸, —S(O)₂C(O)OR⁷, or —P(O)(OR⁷)(OR⁸). In some embodiments, each instance of R¹, R², R³, R⁴, R⁵, and R⁶ is independently —H or optionally substituted C₁₋₁₈ alkyl. In some embodiments, each instance of R¹, R², R³, R⁴, R⁵, and R⁶ is independently —H or optionally substituted C₁₋₆ alkyl. In some embodiments, each instance of R¹, R², R³, R⁴, R⁵, and R⁶ is independently —H or C₁₋₆ alkyl.

In one embodiment, the hydrogen atom is abstracted by an organic radical generated by the non-heme metalloenzyme. In a particular embodiment, the organic radical is generated through homolysis of a bond on a radical precursor. In some embodiments, the radical precursor is coupled to the organic substrate. In some embodiments, the bond on the radical precursor is a halogen-halogen bond, a carbon-halogen bond, a nitrogen-halogen bond, or an oxygen-oxygen bond. In a specific embodiment, the method includes coupling a nucleophile to an organic substrate that contains a C—H bond by contacting the organic substrate with a nucleophile source (M⁺X⁻) containing the nucleophile, a radical precursor, and a non-heme metalloenzyme, thereby converting the organic substrate into a reaction product in which the C—H bond is replaced by a bond between the carbon and the nucleophile group. A general outline for this reaction is provided in SCHEME 2, wherein R—H is the organic substrate, M⁺X⁻ is the nucleophile source, and R—X is the product.

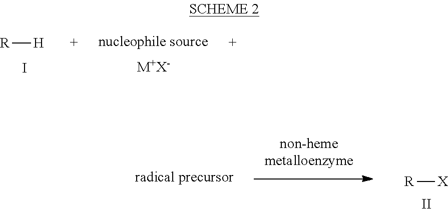

In some embodiments, the C—H bond is an allylic C—H bond, a benzylic C—H bond, a propargylic C—H bond, or an aliphatic C—H bond. In some embodiments, the C—H bond is an aliphatic C—H bond.

In some embodiments, the nucleophile is fluoro, chloro, bromo, iodo, azido, thiocyanate, cyanate, isothiocyanate, isonitrile, cyanide, alkoxylate, thiolate, or a nitrogen-containing heterocycle. In some cases, the nucleophile source has a structure according to Formula (XIX):

M⁺X⁻ (XIX)

wherein M⁺ is Na⁺, K⁺, Cs⁺, or [N(R¹²)₄]⁺; and wherein X⁻ is F⁻, Cl⁻, Br⁻, I⁻, N₃⁻, SCN⁻, CN⁻, NCO⁻, [SR¹³]⁻, or [OR¹³]⁻; wherein each instance of R¹² is independently —H, C₁-C₆ alkyl, or C₁-C₆ haloalkyl, or wherein two instances of R¹² are taken together along with the nitrogen to which they are attached to form a C₂-C₈ heterocycloalkyl; and wherein each instance of R¹³ is independently —H, C₁-C₆ alkyl, or C₁-C₆ haloalkyl. In some embodiments, the nucleophile source has a structure according to any one of Formulas (VIII)—(XVII):

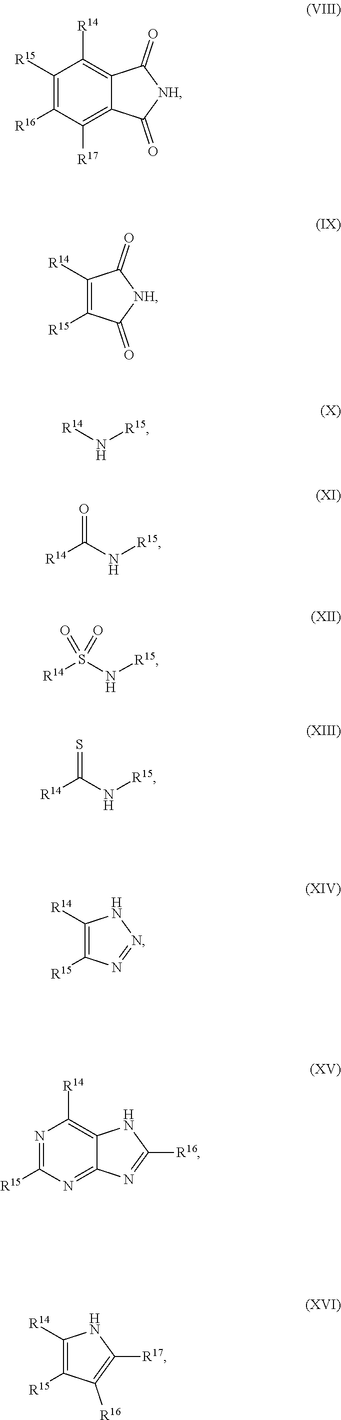

wherein each instance of R¹⁴, R¹⁵, R¹⁶, and R⁷ is independently —H, optionally substituted C₁₋₁₈ alkyl, C₁₋₁₈ polyfluoroalkyl, optionally substituted C₂₋₁₈ alkenyl, optionally substituted C₂₋₁₈ alkynyl, optionally substituted C₆₋₁₀ aryl, optionally substituted 6- to 10-membered heteroaryl, optionally substituted 6- to 10-membered heterocyclyl, cyano, halo, nitro, —NR¹⁸R¹⁹, —BR²¹R²², —SiR¹⁸R¹⁹R²⁰, —C(O)OR¹⁸, —C(O)SR¹⁸, —C(O)NR¹⁸R¹⁹, —C(O)R¹⁸, —C(O)ONR¹⁸R¹⁹, —C(O)NR¹⁸OR¹⁹, —C(O)C(O)OR¹⁸, —S(O)OR¹⁸, —S(O)SR¹⁸, —S(O)NR¹⁸R¹⁹, —S(O)R¹⁸, —S(O)ONR¹⁸R¹⁹, —S(O)NR¹⁸OR¹⁹, —S(O)C(O)OR¹⁸, —S(O)₂OR¹⁸, —S(O)₂SR¹⁸, —S(O)₂NR¹⁸R¹⁹, —S(O)₂R¹⁸, —S(O)₂ONR¹⁸R¹⁹, —S(O)₂NR¹⁸OR¹⁹, —S(O)₂C(O)OR¹⁸, or —P(O)(OR¹⁸)(OR¹⁹); each instance of R¹⁸, R¹⁹, and R²⁰ is independently —H, C₁-C₃ alkyl, or C₁-C₃ haloalkyl; and each instance of R²¹ and R²² is independently —H, C₁-C₃ alkyl, C₁-C₃ haloalkyl, or —OR¹⁸.

In some embodiments, each instance of R¹⁴, R¹⁵, R¹⁶, and R¹⁷ is independently —H or optionally substituted C₁₋₁₈ alkyl. In some embodiments, each instance of R¹⁴, R¹⁵, R¹⁶, and R¹⁷ is independently —H or optionally substituted C₁₋₆ alkyl. In some embodiments, each instance of R¹⁴, R¹⁵, R¹⁶, and R¹⁷ is independently —H or C₁₋₆ alkyl.

In some embodiments, the radical precursor has a structure according to any one of Formulas (I)—(VII):

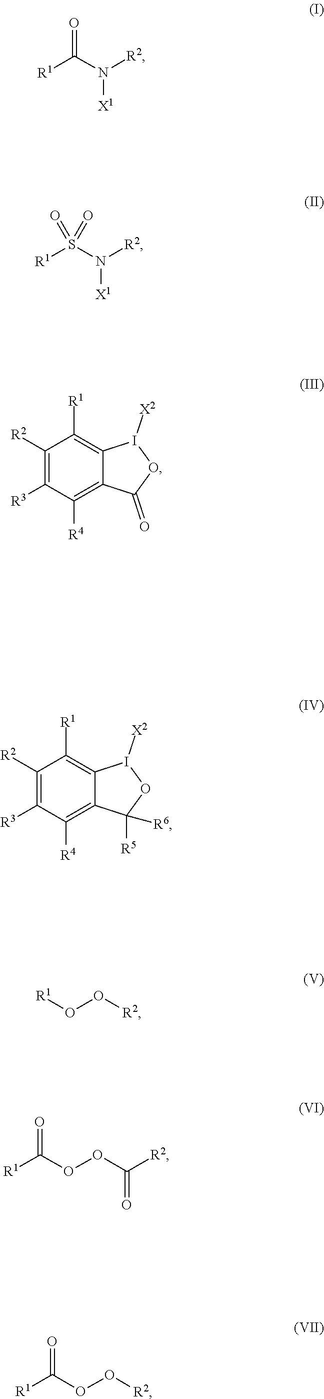

wherein each instance of R¹, R², R³, R⁴, R⁵, and R⁶ is independently the organic substrate, —H, optionally substituted C₁₋₁₈ alkyl, optionally substituted C₁₋₁₈ polyfluoroalkyl, optionally substituted C₂₋₁₈ alkenyl, optionally substituted C₂₋₁₈ alkynyl, optionally substituted C₆₋₁₀ aryl, optionally substituted 6- to 10-membered heteroaryl, optionally substituted 6- to 10-membered heterocyclyl, cyano, halo, nitro, —NR⁷R⁸, —BR¹⁰R¹¹, —SiR⁷R⁸R⁹, —C(O)OR⁷, —C(O)SR⁷, —C(O)NR⁷R⁸, —C(O)R⁷, —C(O)ONR⁷R⁸, —C(O)NR⁷OR⁸, —C(O)C(O)OR⁷, —S(O)OR⁷, —S(O)SR⁷, —S(O)NR⁷R⁸, —S(O)R⁷, —S(O)ONR⁷R⁸, —S(O)NR⁷OR⁸, —S(O)C(O)OR⁷, —S(O)₂OR⁷, —S(O)₂SR⁷, —S(O)₂NR⁷R⁸, —S(O)₂R⁷, —S(O)₂ONR⁷R⁸, —S(O)₂NR⁷OR⁸, —S(O)₂C(O)OR⁷, or —P(O)(OR⁷)(OR⁸);
each instance of R⁷, R⁸, and R⁹ is independently —H, C₁-C₃ alkyl, or C₁-C₃ haloalkyl;
each instance of R¹⁰ and R¹¹ is independently —H, C₁-C₃ alkyl, C₁-C₃ haloalkyl, or —OR⁷;
each instance of X¹ is independently —F, —Cl, —Br, or —I; and
each instance of X² is independently —F, —Cl, or —Br.

In some embodiments, each instance of R¹, R², R³, R⁴, R⁵, and R⁶ is independently —H, optionally substituted C₁₋₁₈ alkyl, optionally substituted C₁₋₁₈ polyfluoroalkyl, optionally substituted C₂₋₁₈ alkenyl, optionally substituted C₂₋₁₈ alkynyl, optionally substituted C₆₋₁₀ aryl, optionally substituted 6- to 10-membered heteroaryl, optionally substituted 6- to 10-membered heterocyclyl, cyano, halo, nitro, —NR⁷R⁸, —BR¹⁰R¹¹, —SiR⁷R⁸R⁹, —C(O)OR⁷, —C(O)SR⁷, —C(O)NR⁷R⁸, —C(O)R⁷, —C(O)ONR⁷R⁸, —C(O)NR⁷OR⁸, —C(O)C(O)OR⁷, —S(O)OR⁷, —S(O)SR⁷, —S(O)NR⁷R⁸, —S(O)R⁷, —S(O)ONR⁷R⁸, —S(O)NR⁷OR⁸, —S(O)C(O)OR⁷, —S(O)₂OR⁷, —S(O)₂SR⁷, —S(O)₂NR⁷R⁸, —S(O)₂R⁷, —S(O)₂ONR⁷R⁸, —S(O)₂NR⁷OR⁸, —S(O)₂C(O)OR⁷, or —P(O)(OR⁷)(OR⁸). In some embodiments, each instance of R¹, R², R³, R⁴, R⁵, and R⁶ is independently —H or optionally substituted C₁₋₁₈ alkyl. In some embodiments, each instance of R¹, R², R³, R⁴, R⁵, and R⁶ is independently —H or optionally substituted C₁₋₆ alkyl. In some embodiments, each instance of R¹, R², R³, R⁴, R⁵, and R⁶ is independently —H or C₁₋₆ alkyl. In some embodiments, each instance of X¹ is independently —F or —Cl. In some embodiments, each instance of X¹ is —F. In some embodiments, each instance of X² is independently —F or —Cl. In some embodiments, each instance of X² is —F.

In some embodiments, the present invention provides a method for coupling a nucleophile group to an organic substrate that contains a C—H bond by contacting the organic substrate with a nucleophile source (M⁺X⁻) containing the nucleophile and a non-heme metalloenzyme, thereby converting the organic substrate to a reaction product in which the C—H bond is replaced by a bond between the carbon and the nucleophile. Contrasting many radical transfer reactions, an N-haloamine of the organic substrate can be stable during the method (e.g. the N-haloamine is not dehalogenated in the presence of the non-heme metalloenzyme and nucleophile source). For example, in some embodiments, the compound containing the organic substrate has a structure according to Formula (XVIII):

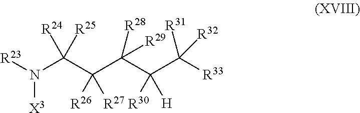

wherein each instance of R²³, R²⁴, R²⁵, R²⁶, R²⁷, R²⁸, R²⁹, R³⁰, R³¹, R³², and R³³ is independently —H, optionally substituted C₁₋₁₈ alkyl, C₁₋₁₈ polyfluoroalkyl, optionally substituted C₂₋₁₈ alkenyl, optionally substituted C₂₋₁₈ alkynyl, optionally substituted C₆₋₁₀ aryl, optionally substituted 6- to 10-membered heteroaryl, optionally substituted 6- to 10-membered heterocyclyl, cyano, halo, nitro, —NR³⁴R³⁵, —BR³⁷R³⁸, —SiR³⁴R³⁵R³⁶, —C(O)OR³⁴, —C(O)SR³⁴, —C(O)NR³⁴R³⁵, —C(O)R³⁴, —C(O)ONR³⁴R³⁵, —C(O)NR³⁴OR³⁵, —C(O)C(O)OR³⁴, —S(O)OR³⁴, —S(O)SR³⁴, —S(O)NR³⁴R³⁵, —S(O)R³⁴, —S(O)ONR³⁴R³⁵, —S(O)NR³⁴OR³⁵, —S(O)C(O)OR³⁴, —S(O)₂OR³⁴, —S(O)₂SR³⁴, —S(O)₂NR³⁴R³⁵, S(O)₂R³⁴, S(O)₂ONR³⁴R³⁵, —S(O)₂NR³⁴OR³⁵, —S(O)₂C(O)OR³⁴, or —P(O)(OR³⁴)(OR³⁵).
each instance of R³⁴, R³⁵, and R³⁶ is independently —H, C₁-C₃ alkyl, or C₁-C₃ haloalkyl;
each instance of R³⁷ and R³⁸ is independently —H, C₁-C₃ alkyl, C₁-C₃ haloalkyl, or —OR³⁴; and
X₃ is —F, —Cl, —Br, or —I.

In such cases, the method may follow a reaction as outlined in SCHEME 3.

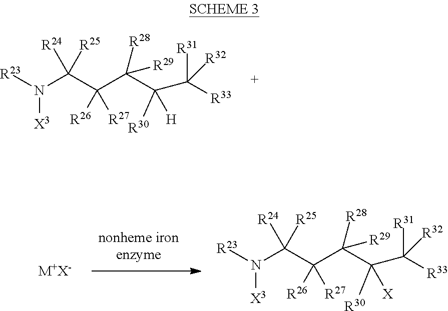

In some embodiments, the C—H bond is an allylic C—H bond, a benzylic C—H bond, a propargylic C—H bond, or an aliphatic C—H bond. In some embodiments, the C—H bond is an aliphatic C—H bond.

In some embodiments, the nucleophile is fluoro, chloro, bromo, iodo, azido, thiocyanate, cyanate, isothiocyanate, isonitrile, cyanide, alkoxylate, thiolate, or a nitrogen-containing heterocycle. In some embodiments, the nucleophile is a halogen or an azide. In some cases, the nucleophile source has a structure according to Formula (XIX):

M⁺X⁻ (XIX),

wherein M⁺ is Na⁺, K⁺, Cs⁺, or [N(R¹²)₄]⁺; and wherein X⁻ is F⁻, Cl⁻, Br⁻, I⁻, N₃⁻, SCN⁻, CN⁻, NCO⁻, [SR¹³]⁻, or [OR¹³]⁻; wherein each instance of R¹² is independently —H, C₁-C₆ alkyl, or C₁-C₆ haloalkyl, or wherein two instances of R¹² are taken together along with the nitrogen to which they are attached to form a C₂-C₈ heterocycloalkyl; and wherein each instance of R¹³ is independently —H, C₁-C₆ alkyl, or C₁-C₆ haloalkyl. In some embodiments, the nucleophile source has a structure according to any one of Formulas (VIII)—(XVII).

In some embodiments, the method includes contacting the organic substrate with the non-heme metalloenzyme, thereby replacing a C—H bond of a carbon with a bond between the carbon and a halogen. In some embodiments, the halogen is coupled to a nitrogen of the organic substrate (e.g., as an N-haloamine) prior to the method. In such cases, the method can transfer the C—H bond hydrogen to the nitrogen of the nitrogen. For example, the method can utilize a compound of Formula (XVIII) and proceed according to SCHEME 4, wherein X³ is transferred from a nitrogen on the organic substrate to a carbon on the organic substrate, and a hydrogen is transferred from the carbon of the organic substrate to the nitrogen of the organic substrate.

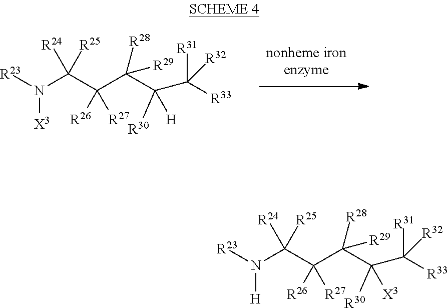

As detailed further herein, the use of non-heme metalloenzymes can provide high degrees of stereochemical control over a reaction. While many radical mechanisms racemize substrates, active site sterics imposed by the non-heme metalloenzyme can impose isomerism upon transition states and reaction intermediates (e.g. H- or X-atom abstracted organic substrates) to achieve asymmetric catalysis. In some cases, a reaction product has an enantiomeric ratio of at least about 60:40, at least about 65:35, at least about 70:30, at least about 75:25, at least about 80:20, at least about 85:15, at least about 90:10, or at least about 95:5. In some cases, the reaction product has an excess of (R)-enantiomers relative to (S)-enantiomers. In some cases, the reaction product has an excess of (S)-enantiomers relative to (R)-enantiomers.

The non-heme metalloenzyme can be an enzyme containing a non-heme metal cofactor. While heme enzymes are unique among natural enzymes in their ability to oxidize stable substrates and stabilize low spin and high valence iron centers (e.g., iron(IV)) that can promote 2-electron oxidation chemistry over controlled one electron radical mechanisms. As disclosed herein, repurposed non-heme metalloenzymes can utilize non-heme metal cofactors to generate and manipulate radical intermediates with high degrees of chemical and stereochemical control. The non-heme metalloenzyme can catalyze the in vitro and in vivo formation of carbon-nitrogen, carbon-sulfur, carbon-carbon, and carbon-halogen bonds by combining different synthetic radical C—H activation mechanisms with metal-mediated bond forming processes.

In some embodiments, the non-heme metalloenzyme includes an iron cofactor, a copper cofactor, a cobalt cofactor, a manganese cofactor, a nickel cofactor, or a chromium cofactor (e.g., the cofactor that mediates a reaction disclosed herein). In some cases, the non-heme metalloenzyme includes an iron cofactor. In some cases, the non-heme metalloenzyme includes a nonnative metal cofactor. For example, the non-heme metalloenzyme can be a non-heme iron enzyme expressed in apo form and loaded with a copper, cobalt, manganese, nickel, or a chromium cofactor. Alternatively, the non-heme metalloenzyme that natively utilizes a non-iron metal cofactor can be repurposed with an iron cofactor for use in a method disclosed herein.

In some embodiments, the non-heme metalloenzyme is an iron(II) enzyme (e.g., contains an iron cofactor with a +2 oxidation state). The non-heme iron enzyme can serve as a catalyst, interconverting between iron(II) and iron(III) states during the method. In particular cases, the non-heme metalloenzyme includes iron(II) that converts to iron (III) upon radical generation (e.g., H- or X-atom abstraction (halogen atom abstraction) from the organic substrate or halogen source) and converts back to iron(II) upon H- or X-atom donation (halogen atom donation) to the substrate or halogen source. In some embodiments, the iron cofactor does not adopt a +4 oxidation state. As iron(IV) can be a strong oxidant, avoiding iron(IV) oxidation states can limit promiscuous oxidation chemistry and side product generation by the iron cofactor.

In some aspects, the methods are performed in the absence of oxygen (i.e., under anoxic or anaerobic conditions) to prevent oxidation or inactivation of the non-heme iron enzyme, to limit radical intermediate quenching, and, in the case of in vivo reactions, to limit aerobic metabolism. As used herein, “absence of oxygen” can denote less than 1000 parts per million (ppm) O₂, less than 500 ppm O₂, less than 400 ppm O₂, less than 300 ppm O₂, less than 200 ppm O₂, less than 100 ppm O₂, less than 50 ppm O₂, less than 25 ppm O₂, less than 10 ppm O₂, or less than 5 ppm O₂ in the atmosphere surrounding a reaction system or dissolved within a reaction system.

In some embodiments, the non-heme metalloenzyme is Sav HppD (SEQ ID NO:1) or a fragment or mutant thereof. In some embodiments, the non-heme metalloenzyme has at least about 70% sequence identity to SEQ ID NO:1 and at least 1 mutation relative to SEQ ID NO:1. In some embodiments, In some embodiments the non-heme metalloenzyme has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO:1. In some cases, the non-heme metalloenzyme has at least one mutation relative to SEQ ID NO:1. In some cases, the at least one mutation includes at least 2, at least 3, at least 4, at least 5, at least 6, at least 7, at least 8, at least 9, at least 10, at least 11, at least 12, at least 13, at least 14, or at least 15 mutations relative to SEQ ID NO:1. In some cases, the non-heme metalloenzyme includes at least one, at least two, at least three, at least four, at least five, at least six, at least seven, at least eight, at least nine, at least ten, at least eleven, at least twelve, at least thirteen, at least fourteen, or all fifteen mutations relative to SEQ ID NO:1 at positions selected from H187, V189, N191, L228, S230, P243, N245, Q255, Q269, H270, F336, E349, F364, L367, and F368. In some cases, the non-heme metalloenzyme includes at least one, at least two, at least three, at least four, at least five, at least six, at least seven, at least eight, at least nine, at least ten, or at least eleven mutations relative to SEQ ID NO:1 selected from V189A, N191A, F216A, S230L, P243A, P243G, N245Q, N245F, Q255A, Q255P, and L367I. In some cases, the at least one mutation diminishes active site volume in the non-heme metalloenzyme.

In some embodiments, the non-heme metalloenzyme has at least at least 98.2%, at least 98.4%, at least 98.6%, at least 98.8%, at least 99%, at least 99.2%, at least 99.4%, or at least 99.6% sequence identity to SEQ ID NO:2 or SEQ ID NO:3. In some embodiments, the non-heme metalloenzyme is Sav HppD Az1 (SEQ ID NO:2) or a fragment or mutant thereof. In some embodiments the non-heme metalloenzyme has at least 98.2%, at least 98.4%, at least 98.6%, at least 98.8%, at least 99%, at least 99.2%, at least 99.4%, or at least 99.6% sequence identity to SEQ ID NO:2. In some embodiments, the non-heme metalloenzyme is Sav HppD Az2 (SEQ ID NO:3) or a fragment or mutant thereof. In some embodiments the non-heme metalloenzyme has at least 98.2%, at least 98.4%, at least 98.6%, at least 98.8%, at least 99%, at least 99.2%, at least 99.4%, or at least 99.6% sequence identity SEQ ID NO:3.

Exemplary non-heme metalloenzymes which can be utilized for the methods of the present invention are listed in TABLE 1. In certain embodiments, the non-heme metalloenzyme is 4-hydroxymandelate synthase from Amycolatopsis orientalis, 4-hydroxyphenylpyruvate dioxygenase from Streptomyces avermitilis, isopenicillin N synthase from Emericella nidulans, 2-hydroxypropylphosphonic acid epoxidase from Streptomyces viridochromogenes, phenylalanine hydroxylase from Chromobacterium violaceum, hercynine oxygenase from Mycolicibacterium thermoresistibile, α-ketoglutarate-dependent dioxygenase AlkB from Escherichia coli, α-ketoglutarate-dependent halogeanse SyrB2 from Pseudomonas syringae, α-ketoglutarate-dependent halogeanse BesD from Streptantibioticus cattleyicolor, α-ketoglutarate-dependent dioxygenase SadA from Burkholderia ambifaria, α-ketoglutarate-dependent dioxygenase Evdo2 from Micromonospora carbonacea, proline cis-4-hydroxylase from Mesorhizobium japonicum, polyoxin hydroxylase from Streptomyces aureochromogenes, or a variant thereof. In certain embodiments, the non-heme metalloenzyme has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to any one of SEQ ID NO:1-16. In some embodiments, the non-heme metalloenzyme has at least one, at least two, at least three, at least four, at least five, at least six, at least seven, at least eight, at least nine, or at least ten mutations relative to any one of SEQ ID NO:1-16.

TABLE 1

| | SEQ | |
|---|---|---|
| Enzyme | ID NO | SEQUENCE |
| Sav HppD | SEQ ID | MTQTTHHTPDTARQADPFPVKGMDAVVFAVGN |
| | NO: 1 | AKQAAHYYSTAFGMQLVAYSGPENGSRETASY |
| | | VLTNGSARFVLTSVIKPATPWGHFLADHVAEHG |
| | | DGVVDLAIEVPDARAAHAYAIEHGARSVAEPYE |
| | | LKDEHGTVVLAAIATYGKTRHTLVDRTGYDGPY |
| | | LPGYVAAAPIVEPPAHRTFQAIDHCVGNVELGR |
| | | MNEWVGFYNKVMGFTNMKEFVGDDIATEYSAL |
| | | MSKVVADGTLKVKFPINEPALAKKKSQIDEYLEF |
| | | YGGAGVQHIALNTGDIVETVRTMRAAGVQFLDT |
| | | PDSYYDTLGEWVGDTRVPVDTLRELKILADRDE |
| | | DGYLLQIFTKPVQDRPTVFFEIIERHGSMGFGKGN |
| | | FKALFEAIEREQEKRGNL |
| Sav HppD Az1 | SEQ ID | MTQTTHHTPDTARQADPFPVKGMDAVVFAVGN |
| | NO: 2 | AKQAAHYYSTAFGMQLVAYSGPENGSRETASY |
| | | VLTNGSARFVLTSVIKPATPWGHFLADHVAEHG |
| | | DGVVDLAIEVPDARAAHAYAIEHGARSVAEPYE |
| | | LKDEHGTVVLAAIATYGKTRHTLVDRTGYDGPY |
| | | LPGYVAAAPIVEPPAHRTFQAIDHCAGNVELGR |
| | | MNEWVGFYNKVMGFTNMKEAVGDDIATEYSAL |
| | | MSKVVADGTLKVKFAIQEPALAKKKSAIDEYLEF |
| | | YGGAGVQHIALNTGDIVETVRTMRAAGVQFLDT |
| | | PDSYYDTLGEWVGDTRVPVDTLRELKILADRDE |
| | | DGYLLQIFTKPVQDRPTVFFEIIERHGSMGFGKGN |
| | | FKAIFEAIEREQEKRGNL |
| Sav HppD Az2 | SEQ ID | MTQTTHHTPDTARQADPFPVKGMDAVVFAVGN |
| | NO: 3 | AKQAAHYYSTAFGMQLVAYSGPENGSRETASY |
| | | VLTNGSARFVLTSVIKPATPWGHFLADHVAEHG |
| | | DGVVDLAIEVPDARAAHAYAIEHGARSVAEPYE |
| | | LKDEHGTVVLAAIATYGKTRHTLVDRTGYDGPY |
| | | LPGYVAAAPIVEPPAHRTFQAIDHCAGAVELGR |
| | | MNEWVGFYNKVMGFTNMKEFVGDDIATEYSAL |
| | | MLKVVADGTLKVKFGIFEPALAKKKSPIDEYLEF |
| | | YGGAGVQHIALNTGDIVETVRTMRAAGVQFLDT |
| | | PDSYYDTLGEWVGDTRVPVDTLRELKILADRDE |
| | | DGYLLQIFTKPVQDRPTVFFEIIERHGSMGFGKGN |
| | | FKAIFEAIEREQEKRGNL |
| Amycolatopsis orientalis | SEQ ID | MQNFEIDYVEMYVENLEVAAFSWVDKYAFAVA |
| 4-hydroxymandelate | NO: 4 | GTSRSADHRSIALRQGQVTLVLTEPTSDRHPAAA |
| synthase | | YLQTHGDGVADIAMATSDVAAAYEAAVRAGAE |
| | | AVRAPGQHSEAAVTTATIGGFGDVVHTLIQRDG |
| | | TSAELPPGFTGSMDVTNHGKGDVDLLGIDHFAIC |
| | | LNAGDLGPTVEYYERALGFRQIFDEHIVVGAQA |
| | | MNSTVVQSASGAVTLTLIEPDRNADPGQIDEFLK |
| | | DHQGAGVQHIAFNSNDAVRAVKALSERGVEFLK |
| | | TPGAYYDLLGERITLQTHSLDDLRATNVLADED |
| | | HGGQLFQIFTASTHPRHTIFFEVIERQGAGTFGSS |
| | | NIKALYEAVELERTGQSEFGAARR |
| Streptomyces avermitilis | SEQ ID | MTQTTHHTPDTARQADPFPVKGMDAVVFAVGN |
| 4-hydroxyphenylpyruvate | NO: 5 | AKQAAHYYSTAFGMQLVAYSGPENGSRETASY |
| dioxygenase | | VLTNGSARFVLTSVIKPATPWGHFLADHVAEHG |
| | | DGVVDLAIEVPDARAAHAYAIEHGARSVAEPYE |
| | | LKDEHGTVVLAAIATYGKTRHTLVDRTGYDGPY |
| | | LPGYVAAAPIVEPPAHRTFQAIDHCVGNVELGR |
| | | MNEWVGFYNKVMGFTNMKEFVGDDIATEYSAL |
| | | MSKVVADGTLKVKFPINEPALAKKKSQIDEYLEF |
| | | YGGAGVQHIALNTGDIVETVRTMRAAGVQFLDT |
| | | PDSYYDTLGEWVGDTRVPVDTLRELKILADRDE |
| | | DGYLLQIFTKPVQDRPTVFFEIIERHGSMGFGKGN |
| | | FKALFEAIEREQEKRGNL |
| Emericella nidulans | SEQ ID | MGSVSKANVPKIDVSPLFGDDQAAKMRVAQQID |
| isopenicillin N synthase | NO: 6 | AASRDTGFFYAVNHGINVQRLSQKTKEFHMSITP |
| | | EEKWDLAIRAYNKEHQDQVRAGYYLSIPGKKAV |
| | | ESFCYLNPNFTPDHPRIQAKTPTHEVNVWPDETK |
| | | HPGFQDFAEQYYWDVFGLSSALLKGYALALGKE |
| | | ENFFARHFKPDDTLASVVLIRYPYLDPYPEAAIKT |
| | | AADGTKLSFEWHEDVSLITVLYQSNVQNLQVET |
| | | AAGYQDIEADDTGYLINCGSYMAHLTNNYYKAP |
| | | IHRVKWVNAERQSLPFFVNLGYDSVIDPFDPREP |
| | | NGKSDREPLSYGDYLQNGLVSLINKNGQT |
| Streptomyces | SEQ ID | MSNTKTASTGFAELLKDRREQVKMDHAALASLL |
| viridochromogenes 2- | NO: 7 | GETPETVAAWENGEGGELTLTQLGRIAHVLGTSI |
| hydroxypropylphosphonic | | GALTPPAGNDLDDGVIIQMPDERPILKGVRDNVD |
| acid epoxidase | | YYVYNCLVRTKRAPSLVPLVVDVLTDNPDDAKF |
| | | NSGHAGNEFLFVLEGEIHMKWGDKENPKEALLP |
| | | TGASMFVEEHVPHAFTAAKGTGSAKLIAVNF |
| Chromobacterium | SEQ ID | MNDRADFVVPDITTRKNVGLSHDANDFTLPQPL |
| violaceum phenylalanine | NO: 8 | DRYSAEDHATWATLYQRQCKLLPGRACDEFME |
| hydroxylase | | GLERLEVDADRVPDFNKLNQKLMAATGWKIVA |
| | | VPGLIPDDVFFEHLANRRFPVTWWLREPHQLDY |
| | | LQEPDVFHDLFGHVPLLINPVFADYLEAYGKGG |
| | | VKAKALGALPMLARLYWYTVEFGLINTPAGMRI |
| | | YGAGILSSKSESIYCLDSASPNRVGFDLMRIMNTR |
| | | YRIDTFQKTYFVIDSFKQLFDATAPDFAPLYLQLA |
| | | DAQPWGAGDVAPDDLVLNAGDRQGWADTEDV |
| hercynine oxygenase from | SEQ ID | MTGVAVPHRAELARQLIDARNRTLRLVDFDDAE |
| Mycolicibacterium | NO: 9 | LRRQYDPLMSPLVWDLAHIGQQEELWLLRGGDP |
| thermoresistibile, | | RRPGLLEPAVEQLYDAFVHPRASRVHLPLLSPAQ |
| | | ARRFCATVRSAVLDALDRLPEDADTFAFGMVVS |
| | | HEHQHDETMLQALNLRSGEPLLGSGTALPPGRP |
| | | GVAGTSVLVPGGPFVLGVDLADEPYALDNERPA |
| | | HVVDVPAFRIGRVPVTNAEWRAFIDDGGYRQRR |
| | | WWSDAGWAYRCEAGLTAPQFWNPDGTRTRFG |
| | | HVEDIPPDEPVQHVTYFEAEAYAAWAGARLPTEI |
| | | EWEKACAWDPATGRRRRYPWGDAAPTAALANL |
| | | GGDALRPAPVGAYPAGASACGAEQMLGDVWE |
| | | WTSSPLRPWPGFTPMIYQRYSQPFFEGAGSGDYR |
| | | VLRGGSWAVAADILRPSFRNWDHPIRRQIFAGVR |
| | | LAWDVDRQTARPGPVGGC |
| Escherichia coli α- | SEQ ID | MLDLFADAEPWQEPLAAGAVILRRFAFNAAEQLI |
| ketoglutarate-dependent | NO: 10 | RDINDVASQSPFRQMVTPGGYTMSVAMTNCGHL |
| dioxygenase AlkB | | GWTTHRQGYLYSPIDPQTNKPWPAMPQSFHNLC |
| | | QRAATAAGYPDFQPDACLINRYAPGAKLSLHQD |
| | | KDEPDLRAPIVSVSLGLPAIFQFGGLKRNDPLKRL |
| | | LLEHGDVVVWGGESRLFYHGIQPLKAGFHPLTID |
| | | CRYNLTFRQAGKKE |
| Pseudomonas syringae α- | SEQ ID | MSKKFALTAEQRASFEKNGFIGPFDAYSPEEMKE |
| ketoglutarate-dependent | NO: 11 | TWKRTRLRLLDRSAAAYQDLDAISGGTNIANYD |
| halogeanse SyrB2 | | RHLDDDFLASHICRPEICDRVESILGPNVLCWRTE |
| | | FFPKYPGDEGTDWHQADTFANASGKPQIIWPENE |
| | | EFGGTITVWTAFTDANIANGCLQFIPGTQNSMNY |
| | | DETKRMTYEPDANNSVVKDGVRRGFFGYDYRQ |
| | | LQIDENWKPDEASAVPMQMKAGQFIIFWSTLMH |
| | | ASYPHSGESQEMRMGFASRYVPSFVHVYPDSDHI |
| | | EEYGGRISLEKYGAVQVIGDETPEYNRLVTHTTR |
| | | GKKFEAV |
| Streptantibioticus | SEQ ID | MCAPLEKDDIRRLSQAFHRFGIVTVTELIEPHTRK |
| cattleyicolor α- | NO: 12 | LVRAEADRLLDQYAERRDLRLATTDYTRRSMSV |
| ketoglutarate-dependent | | VPSETIAANSELVTGLYAHRELLAPLEAIAGERLH |
| halogeanse BesD | | PCPKADEEFLITRQEQRGDTHGWHWGDFSFALI |
| | | WVLQAPPIDVGGLLQCVPHTTWDKASPQINRYL |
| | | VENPIDTYHFESGDVYFLRTDTTLHRTIPLREDTT |
| | | RIILNMTWAGERDLSRKLAADDRWWDNAEVSA |
| | | ARAI |
| Burkholderia ambifaria α- | SEQ ID | MQHTYPAQLMRFGTAARAEHMTIAAAIHALDA |
| ketoglutarate-dependent | NO: 13 | DEADAIVMDIVPDGERDAWWDDEGFSSSPFTKN |
| dioxygenase SadA | | AHHAGIVATSVTLGQLQREQGDKLVSKAAEYFG |
| | | IACRVNDGLRTTRFVRLESDALDAKPLTIGHDYE |
| | | VEFLLATRRVYEPFEAPFNFAPHCDDVSYGRDTV |
| | | NWPLKRSFPRQLGGFLTIQGADNDAGMVMWDN |
| | | RPESRAALDEMHAEYRETGAIAALERAAKIMLK |
| | | PQPGQLTLFQSKNLHAIERCTSTRRTMGLFLIHTE |
| | | DGWRMFD |
| Micromonospora | SEQ ID | MDAMEVVGTIDHRDREEFRSRGFAILPQVASESE |
| carbonacea α- | NO: 14 | VAWLRQAYDRLFVRRATPGAEDFYDIAGQRDRE |
| ketoglutarate-dependent | | GPPLLPQIIKPEKYVPELLDSPHFARCRSIASAFLD |
| dioxygenase Evdo2 | | MAEEELEFYGHAILKPPRYGAPTPWHQDEAYMD |
| | | PRWRRRGLSIWTTLDEATVESGCLHYLPGGHRG |
| | | PVLPHHHIDNDDRIRGLMTDDVDPTSAVACPLAP |
| | | GGAVVHDFRTPHYAGPNLTDQPRRAYVLVFMS |
| | | APAEVADPEPRPWMDWG |
| Mesorhizobium japonicum | SEQ ID | MTTRILGVVQLDQRRLTDDLAVLAKSNFSSEYSD |
| Proline cis-4-hydroxylase | NO: 15 | FACGRWEFCMLRNQSGKQEEQRVVVHETPALA |
| | | TPLGQSLPYLNELLDNHEDRDSIRYARIIRISENAC |
| | | IIPHRDYLELEGKFIRVHLVLDTNEKCSNTEENNI |
| | | FHMGRGEIWFLDASLPHSAGCFSPTPRLHLVVDI |
| | | EGTRSLEEVAINVEQPSARNATVDTRKEWTDETL |
| | | ESVLGFSEIISEANYREIVAILAKLHFFHKVHCVD |
| | | MYGWLKEICRRRGEPALIEKANSLERFYLIDRAA |
| | | GEVMTY |
| Streptomyces | SEQ ID | MLTRPTAALSSPADITGDLVRTGFSMVPGSDMR |
| aureochromogenes | NO: 16 | VPAALQDSLKTLAASYDDLPADPYLPDGGNYRY |
| polyoxin hydroxylase | | RRHTRYTWRPATGELLVADNPGYFQTVENNAFA |
| | | GGQWRKYEELTDEVREGAFLTALIDENVGRLPLP |
| | | EVEQWAVQVHCVRIVARDDAQGRPTPEGVHRD |
| | | GCTYVSLHMVNRHNISGGRTSVYTPEHELITEKV |
| | | FTDCLDSFFGDDPRVRHGVADVSVADPSLGEGT |
| | | RDMLLMSYDPM |

In certain embodiments, the non-heme metalloenzyme is a non-heme metalloenzyme listed in TABLE 6 or a mutant thereof.

TABLE 6

| Enzyme Name | Protein ID |
|---|---|
| AaSOR | uniprot P29082 |
| AgAO | P46881 |
| AkbC | uniprot Q6REQ5 |
| AlkB | P05050, pBLAST |
| | Sequence ID: |
| | WP_000884971.1 |
| AoDHP | uniprot Q9NAV7 |
| ApPgb LQ | uniprot Q9YFF4 |
| ArCTD | uniprot Q9F103 |
| AroG | uniprot 053512 |
| AsqJ | Q5AR53, pBLAST |
| | Sequence: |
| | XP_682496.1 |
| AtLDOX | uniprot Q96323 |
| AviO1 | uniprot Q93KW4 |
| BaP4H | Uniprot Q81LZ8 |
| CglAlcOx | E3QHV8 |
| Cj LPMO | uniprot B3PJ79 res 37- |
| | 216 |
| CkoPE | uniprot B1L4V6 |
| CotA_NG | uniprot P07788 |
| CylC | Uniprot K7S6E6 |
| CYP2C9_T301G_{—} | uniprot P11712 |
| C435S | |
| CYP101_T252G-C358S | Uniprot P00183 |
| CYP119 | uniprot Q55080 |
| CYP119_T213G_C317S | uniprot Q55080 |
| CYP153A7_T259G- | Uniprot Q5F4D9 |
| C366S | |
| CytC3 | Uniprot DOVX22, |
| | pBLAST Sequence |
| | ID: 3GJA_A |
| DAD | uniprot Q9REI7 |
| DaP4H-NHis | pBLAST Sequence |
| | ID: AIS76464.1 |
| DaP4H_L139S | pBLAST Sequence |
| | ID: ANH21194.1 |
| EcMenD | uniprot P17109 |
| EfCBM33A | uniprot Q838S1 |
| EFE | Uniprot Q549K5; |
| | pBLAST Sequence |
| | ID: WP_054082735.1 |
| EgtB | uniprot G7CFI3 |
| EvdO1 | uniprot |
| | A0A0M3KL03 |
| EvdO2 | A0A0M3KL01, |
| | pBLAST Sequence |
| | ID: 4XAB_A |
| EvdO2-pET28a(+) | uniprot |
| | A0A0M3KL01 |
| GLO1 | uniprot Q68RJ8 |
| gloA | uniprot P0AC81 |
| GriE_pET28a | A0A0E3URV8 |
| GYG1 | uniprot P13280 |
| hepD_pET28a | Q51W40 |
| HMS_pET28a | 052791 |
| Hth cyt c | uniprot P15452 |
| HygX | uniprot Q2MFS1 |
| IPNS_pET28a | P05326 |
| Jd LPMO | uniprot C7R410 res |
| | 32-173 |
| LapB | uniprot Q7WYF5 |
| LdoA | pBLAST Sequence |
| | ID: WP_012408787.1 |
| LDOX | Q96323, pBLAST |
| | Sequence ID: |
| | NP_001031700.1 |
| M35_CStrep3 | Unitpro POCS93 |
| M35_CTwinStrep | Unitpro POCS93 |
| MaPgb C101S | uniprot Q8TLY9 |
| Mb VA | uniprot P02185 |
| MerB | uniprot P77072 |
| MIP4H-1 | pBLAST Sequence |
| | ID: WP_010913924.1 |
| mtaD | uniprot Q9X034 |
| MxPDO1b | uniprot Q1D4C9 |
| Ne NitroCyan | uniprot Q820S6 |
| NeSOR | uniprot Q74MF3 |
| Nmar1307 | uniprot A9A2G4 |
| Opd | uniprot Q9NAV7 |
| P411 CHA | uniprot P14779 |
| P450 BM3 | Uniprot P14779 |
| PaAzu DGA | uniprot P00282 |
| PaAzu DGE | uniprot P00282 |
| PaAzu DGQ | uniprot P00282 |
| PaAzu HGA | uniprot P00282 |
| PaAzu HGE | uniprot P00282 |
| PaAzu HGQ | uniprot P00282 |
| PaKae1 | uniprot Q9UXT7 |
| pckA | uniprot A6VKV4 |
| PcTE | uniprot O50580 |
| pddABC_AA | uniprot Q59470 |
| pduCDE | uniprot A0A0H3H0N6; |
| | A0A0H3H347; |
| | A0A0H3H105 |
| PH0974 | uniprot 058691 |
| PHHA | Uniprot P30967 |
| plu4264 | uniprot Q7MZL9 |
| PnpC | uniprot C6FI44 |
| PolL | pBLAST Sequence ID: |
| | ABX24492.1 |
| PpPDO2 | uniprot A5VWI3 |
| Psf4_pET-29b(+) | uniprot Q9JN69 |
| PsHPPD_pET28a | uniprot Q53586 |
| Rgl cyt c | uniprot P00080 |
| Rma cyt c | uniprot B3FQS5 |
| Rma cyt c TDE | uniprot B3FQS5 |
| Rma NOD Y32G | uniprot D0MGT2 |
| rocF | uniprot P53608 |
| RPA4178 | uniprot Q6N272 |
| Rusticyanin | uniprot POC918 |
| SadA_D157G | pBLAST Sequence ID: |
| | WP_011660927.1, |
| | Uniprot Q0B2N4 |
| SadA_D157G_pQTEV | uniprot Q0B2N4 |
| Sav HppD | uniprot Q53586 |
| Sfri_3296 | uniprot Q07XY2 |
| SlBesD | WP_030791981.1 |
| Sli LPMO | uniprot D6EWM4 res |
| | 30 - 201 |
| SmP4H-1 | pBlast Sequence ID: |
| | WP_010970414.1 |
| SpP4H type II | pBLAST Sequence ID: |
| | O09345.1 |
| StrP4H-II_pET28a | uniprot O09345 |
| SwHalB | uniprot A0A1HOBKU7 |
| swHppE | uniprot Q56185 |
| swHppE AA | uniprot Q56185 |
| swHPPE-pET-29b(+) | Q56185 |
| SyrB2_pET28a | Q9RBY6 |
| TaqFBP | uniprot Q9RHA2 |
| TC5S | uniprot Q70AC7 |
| TM0416 | uniprot Q9WYP7 |
| TM1602 | uniprot Q9X1T8 |
| TM_0820 | uniprot Q9WZS7 |
| TM_1162 | uniprot Q9XOP5 |
| TM_1287 | uniprot Q9X113 |
| TPH1 | uniprot p70080 |
| Tt Laccase | uniprot Q72HW2 |
| TtHCS | uniprot 087198 |
| UndA_pET22b | Q4K8M0 |
| vCPH | Uniprot Q84406 |
| vioC_pET28a | Q6WZBO |
| VmoLac | uniprot FOQXN6, PDB: |
| | 4RE0 |
| WelO5 | A0A067YX61, |
| | pBLAST Sequence ID: |
| | 5J4R_A |
| YjbI Y25I T45A Q49A | uniprot 031607 |

As a practical matter, whether any particular nucleic acid molecule or polypeptide is at least, for example, 80%, 85%, 90%, 95%, 96%, 97%, 98% or 99% identical to a nucleotide or peptide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245.) In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

In a further aspect, the present invention provides a composition that includes a non-heme metalloenzyme, an organic substrate comprising a C—H bond, and one or more of a halogen source, a nucleophile source, and a radical precursor as detailed herein.

The present invention further discloses targeted, guided, and directed evolution to develop and enhance enzyme-based catalysts for C—H bond functionalization reactions not previously present in biology. In some cases, the non-heme metalloenzyme includes at least one mutation relative to a wild-type enzyme. In some cases, the mutation increases the hydrophobicity of the active site (e.g., replaces a protic amino acid residue with an aprotic amino acid residue). In some cases, the mutation increases volume of the active site.

In some embodiments, the engineered non-heme iron proteins catalyze carbon-nitrogen, carbon-sulfur, carbon-carbon, and carbon-halogen bond formation with a total turnover number (TTN) over 10000 and enantiomeric excess (ee) up to 94%. Carbon-hydrogen bond functionalization (e.g., C—H functionalization and/or C(sp³)-H functionalization) is a type of reaction in which a carbon-hydrogen bond is cleaved and replaced with a carbon-Y bond (where Y can be carbon, oxygen, sulfur, nitrogen, or a halogen). The term can imply that a transition metal is involved in the C—H cleavage process. Halogens can include fluorine, chlorine, bromine, iodine, astatine, and/or tennessine.

Further disclosed herein are new biocatalysts to perform a non-natural C(sp³)-H azidation reaction. Current synthetic approaches for this reaction are limited in turnovers and enantioselectivity, and often require an acidic azide source to complete the reaction. These limitations were overcome by leveraging the genetic tunability and high catalytic efficiency of multiple metalloenzymes, including a number of non-heme iron enzymes. As detailed further in the examples below, azidation of an N-fluoroamide substrate 1NF was achieved with a panel of nine functionally diverse non-heme iron enzymes under whole-cell conditions. Among the metalloenzymes that were tested, a (4-hydroxyphenyl)pyruvate dioxygenase from Streptomyces avermitilis (Sav HppD) provided the desired azidation product with a total turnovers (TTN) of greater than 100, an enantiomeric ratio (e.r.) of greater than 3:2, and a chemoselectivity of greater than 4:1 for azidation over fluorination product.

Metalloenzymes are a broad group of enzymes that use a metal cation as a cofactor in the enzyme active site. The enzymes promote a diverse range of reactions including hydrolytic processes and oxidation/reductions. Metalloenzymes can include, but are not limited to, non-heme iron enzymes. Metalloenzymes can be reprogrammed and/or modified to select variants suitable for the methods disclosed herein. Suitable metalloenzyme variants can include enantioselective variants. Metalloenzymes suitable for use in the methods disclosed herein include SEQ ID NOS:1-16, metalloenzymes listed in TABLE 6, or mutants thereof.

The method can include use of a reactive radical (X·) to activate C(sp³)-H bond via hydrogen atom transfer (HAT) and the interception of the resulting carbon-centered radical by a redox-reactive metal complex. In some embodiments, a reactive radical (X·) can be a nitrogen radical (N·) and/or an oxygen radical (O·).

In some embodiments, for example, a reprogrammed non-heme iron enzyme can mediate a radical relay process via an initial substrate activation at a Fe(II) center to generate a reactive amidyl radical for HAT and subsequent transfer of a Fe(III)-bound ligand to a carbon-centered radical ring.

In some embodiments, the methods provided herein can include installation of chemically and/or medically relevant moieties such as, but not limited to, azide, chlorine, nitrile, thiocyanate, nitro, or trifluoromethyl.

Accordingly, provided herein are also expanded biocatalysts for drug synthesis and discovery. The methods provided herein broaden the scope of biosynthesis and provide powerful biocatalytic toolbox for late-stage molecular editing of complicated bioactive molecules. For example, biocatalysts (e.g., reprogrammed metalloenzymes) can be used for a variety of industrial applications including drug discovery and synthesis, and sustainable chemical production.

In the preceding description, specific details have been set forth in order to provide a thorough understanding of example implementations of the invention described in the disclosure. However, it will be apparent that various implementations may be practiced without these specific details. For example, circuits, systems, networks, processes, and other components may be shown as components in block diagram form in order not to obscure the example implementations in unnecessary detail. In other instances, well-known circuits, processes, algorithms, structures, and techniques may be shown without unnecessary detail in order to avoid obscuring the examples. The description of the example implementations will provide those skilled in the art with an enabling description for implementing an example of the invention, but it should be understood that various changes may be made in the function and arrangement of elements without departing from the spirit and scope of the invention. Accordingly, the present invention is intended to embrace all such alterations, modifications, and variations that fall within the scope of the appended claims.

### EXAMPLES

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Experimental Methods

### Reagents

Unless otherwise noted, all chemicals and reagents were obtained from commercial suppliers (Sigma-Aldrich, Alfa Aesar, Acros, AA Blocks, Combi-Blocks) and used without further purification. Silica gel chromatography was carried out using SiliaFlash Irregular Silica Gels F60, 40-63 m. 60 Å. ¹H and ¹³C NMR were recorded on either a Bruker Avance 300, 400 or III HD 400 MHz spectrometer. Chemical shifts (δ) are reported in ppm downfield from tetramethylsilane, using the solvent resonance as the internal standard (¹H NMR: δ=7.26, ¹³C NMR: δ=77.4 for CDCl³). Sonication was performed using a Fisherbrand Model 120 Sonic Dismembrator. Chemical reactions were monitored using thin layer chromatography (Merck 60 gel plates) using a UV-lamp for visualization. Gas chromatography-mass spectrometry (GC-MS) analyses were carried out using an Agilent 5977B GC/MSD system and HP-5MS UI column (30.0 m×0.25 mm) with the following oven temperature setting (helium flow 1 ml/min): Initial: 110° C. (hold 0 min); Ramp 1: 110-160° C. (20° C./min, hold 0 min); Ramp 2: 160-225° C. (15° C./min, hold 0 min); Ramp 3: 225-270° C. (30° C./min, hold 4 min). Analytical chiral normal-phase HPLC analyses were performed using an Agilent 1260 series instrument with i-PrOH and hexanes as the mobile phase. Reverse-phase high-performance liquid chromatography-mass spectrometry (LC-MS) analysis was carried out using Agilent 1260 series instruments and Agilent 1260 LC/MSD iQ series instruments. Semi-preparative HPLC was performed using an Agilent XDB-C18 column (9.4×250 mm). Column chromatography was performed on a Biotage Isolera One system using Sfar Silica HC-High Capacity 20 m columns. Plasmid pET22b(+) was used as a cloning vector, and cloning was performed using Gibson assembly (27). Cells were grown using Luria-Bertani (LB) medium or terrific broth (TB) medium (RPI Research). T5 exonuclease, Phusion polymerase, and Taq ligase were purchased from New England Biolabs (NEB, Ipswich, MA). Potassium phosphate buffer (pH 7.4) was used as a buffering system for whole cells, lysates, and purified proteins, unless otherwise specified.

### Generation of Enzyme Variants

All protein variants described in this paper were cloned and expressed using the pET-22b(+) vector or pET-28a(+) vector. The genes encoding non-heme iron proteins used in this work were obtained as a single gBlock (Twist Bioscience), codon-optimized for E. coli, and cloned using Gibson assembly into pET-22b(+) between restriction sites NdeI and XhoI in frame with a C-terminal 6×His-tag or into pET-28a(+) between restriction sites NdeI and BamHI in frame with an N-terminal 6×His-tag. This plasmid was transformed into E. cloni® EXPRESS BL21(DE3) cells (Lucigen).

### Enzyme Expression

200 mL TBₐₘₚ in a 1 L flask was inoculated with an overnight culture (2 mL in LBₐₘₚ) of recombinant E. cloni® EXPRESS BL21(DE3) cells containing a pET-22b(+) plasmid encoding the non-heme iron enzyme variant. The culture was shaken at 37° C. and 240 rpm until the OD₆₀₀ was 0.7 (approximately 2 hours). The culture was placed on ice for 20 minutes, and isopropyl β-D-1-thiogalactopyranoside (IPTG) was added to final concentrations of 1 mM. The incubator temperature was reduced to 20.5° C., and the culture was allowed to shake for 24 hours at 180 rpm. Cells were harvested by centrifugation (4° C., 15 min, 4,000×g) and the cell pellet was resuspended in potassium phosphate buffer (pH 7.4).

### Library Construction

Site-saturation mutagenesis libraries were generated using a modified QuikChange mutagenesis protocol using Phusion® High-Fidelity DNA Polymerase (New England Biolabs). The PCR products were digested with DpnI, gel purified, and the gaps were repaired using Gibson Mix™ (27). Without further purification, 1 μL of the Gibson product was used to transform 50 μL of electrocompetent Escherichia coli BL21 E. cloni (Lucigen) cells. Random mutagenesis was achieved with error-prone PCR using Taq polymerase (New England Biolabs) with a MnCl₂ concentration of 300 μM.

### Library Screening

Single colonies were picked with toothpicks off of LBₐₘₚ agar plates and grown in deep-well (2 mL) 96-well plates containing LBₐₘₚ (400 μL) at 37° C., 240 rpm shaking. After 16 hours, 50 μL aliquots of these overnight cultures were transferred to deep-well 96-well plates containing TBₐₘₚ (1 mL) using a 12-channel Eppendorf Xplorer® plus electronic pipettor. Glycerol stocks of the libraries were prepared by mixing cells in LBₐₘₚ (100 μL) with 50% v/v glycerol (100 μL). Glycerol stocks were stored at −80° C. in 96-well microplates. Growth plates were allowed to shake for 3 hours at 37° C., 240 rpm shaking. The plates were then placed on ice for 30 min. Cultures were induced by adding 10 μL of a solution containing 100 mM isopropyl β-D-1-thiogalactopyranoside (IPTG). The incubator temperature was reduced to 20.5° C., and the induced cultures were allowed to shake for 24 hours (230 rpm). Cells were pelleted (4,500×g, 5 min, 4° C.), resuspended in 400 μL potassium phosphate buffer (pH 7.4), and the plates containing the cell suspensions were transferred to an anaerobic chamber. To deep-well plates of cell suspensions were added sodium azide (10 μL per well, 1.0 M in water), ferrous ammonium sulfate (10 μL per well, 100 mM in water), and the N-fluoroamide model substrate (10 μL per well, 400 mM in dimethoxyethane (DME)). The plates were sealed with aluminum sealing tape and shaken at 680 rpm overnight in the chamber. The plates were then removed from the chamber and analyzed via the high-throughput (HTS) screening assay described in section (E). Hits from library screening were confirmed by small-scale biocatalytic reactions, as described in section (H).

### High-Throughput (HTS) Fluorescent Detection of Azidation Product in 96-Well Plate

Following an azidation reaction, 400 μL of N,N-dimethylformamide (DMF) was added to each well and the plate was incubated for 1 hour. The plate was then centrifuged to remove the insolubles. From each well, 5 μL of the supernatant was transferred to a 96-well black fluorescence plate (Caplugs Evergreen) containing 195 μL of 25% aqueous solution of DMF with 77 μM CuSO₄, 154 μM BTTAA ligand (Click Chemistry Tools), 5.1 mM ascorbic acid, 25.6 mM KPᵢ (pH 7.4), and 103 μM of fluorogenic alkyne probe 4-ethynyl-N-ethyl-1,8-naphthalimide (28). The fluorescence plate was incubated and the formation of the fluorescent triazole product was monitored by a TECAN Spark plate reader outfitted with a plate stacker (excitation wavelength, 357 nm: emission wavelength 462 nm; bandwidth, 20 nm). Validation of hit wells was further investigated by GC-MS. Hits from library screening were confirmed by small-scale biocatalytic reactions, as described in section (H).

### Cell Lysate Preparation

Cell lysates were prepared as follows: E. coli cells expressing non-heme iron enzyme variants were pelleted (4,000×g, 5 min, 4° C.), resuspended in potassium phosphate buffer and adjusted to the appropriate OD₆₀₀. Cells were lysed by sonication (5 minutes, 5 seconds on, 5 seconds off, 40% duty cycle) for two times, aliquotd into 2 mL microcentrifuge tubes, and the cell debris was removed by centrifugation for 10 min (14,000×g, 4° C.). The supernatant was sterile filtered through a 0.45 μm cellulose acetate filter, and the concentration of protein lysate was determined using the described in section (G). Using this protocol, the protein concentrations we typically observed for OD₆₀₀=10 lysates are in the 5-10 μM range for sav HppD and its variants.

### Protein Concentration Determination in Cell Lysates

The quantity of His-tagged non-heme iron enzymes in cell lysates was determined using the His-tag protein ELISA kit according to the manufacturer's instructions (AKR-130 Cell Biolabs, San Diego, CA). Using this protocol, the protein concentrations we typically observed for OD₆₀₀=10 lysates were in the 5-10 μM range for wild-type Sav HppD and its variants.

Small-Scale Biotransformations Using Whole E. coli Cells

In a typical experiment, ferrous ammonium sulfate (20 μL, 100 mM in water), sodium azide (20 μL, 1 M in water), and N-fluoroamide substrate (20 μL, 1.5 M in DME) were added to E. coli harboring non-heme iron enzyme variant (400 μL, adjusted to the appropriate OD₆₀₀) in a 2 mL screw top GC vial in an anaerobic chamber. The vial was capped and shaken at 680 rpm at room temperature for 24 hours. At the end of the reaction, the vial was opened and the reaction was quenched with 6 mL of a hexanes/ethylacetate solution (4:6 v/v) of internal standard 1,2,3-trimethoxybenzene (0.5 mM final concentration). The reaction mixture was transferred to a 15 mL centrifuge tube, vortexed (10 seconds, 3 times), then centrifuged (10,500×g, 5 min) to completely separate the organic and aqueous layers. An aliquot (200-300 μL) of the organic layer was used for product quantification via GCMS and enantioselectivity via chiral HPLC or chiral GC. The total turnover numbers (TTNs) reported are calculated with respect to non-heme iron enzymes expressed in E. coli and represent the total number of turnovers obtained from the catalyst under the stated reaction conditions.

### Protein Purification

Protein expression was conducted following the protocols detailed in section (B). E. coli cells expressing non-heme iron enzyme variants were pelleted (4,000×g, 5 min, 4° C.) and stored at −20° C. for at least 24 hours. The cell pallet was then resuspended in 50 mM KPi buffer containing 100 mM NaCl and 20 mM imidazole (pH 7.5 at 25° C.) (10 mL buffer per gram of cell pellet). Cells were lysed by sonication (5 minutes, 5 seconds on, 5 seconds off, 40% duty cycle) for two times and the cell debris was removed by centrifugation for 10 min (10,300×g, 4° C.). The supernatant was sterile filtered through a 0.45 μm cellulose acetate filter and purified using a 5 mL Ni-NTA column (HisTrap HP, Cytiva) using an ÄKTA start protein purification system (Cytiva). The proteins were eluted from the column by running a gradient from 20 to 500 mM imidazole over 10 column volumes. Fractions containing purified proteins were detected by SDS-PAGE, pooled and concentrated using Millipore® centrifugal filter. The protein solution was dialyzed first against 1 L of buffer with 10 mM EDTA in 50 mM KPi (pH 7.5 at 25° C.), and then two times against 1 L of 50 mM KPi. Final concentration was measured by absorbance at 280 nm using a NanoDrop spectrophotometer. The theoretical extinction coefficients (M⁻¹ cm⁻¹) used for Say HppD and its variants were calculated using ExPASy Bioinformatics Resources Portal.

### Determination of Enantioselectivity

All enantiomeric ratio (e.r.) values of enzymatically synthesized azidation products were determined using normal phase chiral HPLC. The absolute configuration of enzymatically synthesized azidation product 1N was determined to be S via X-ray crystallography. The absolute configurations of all other azidation products were inferred by analogy, assuming the facial selectivity of the C—N₃ bond forming step remains the same as that of 1N. Each chiral determination of the enzymatic product was performed along with the chiral HPLC analysis of the corresponding racemic standard to confirm the retention time of both enantiomers.

### Preparation of Whole-Cell Suspensions for Azidation Reactions

Two hundred milliliter TBₐₘₚ in a one-liter flask was inoculated with an overnight culture (2 mL in LBₐₘₚ) of recombinant E. cloni® EXPRESS BL21(DE3) cells containing a pET22b(+) plasmid encoding the non-heme iron enzyme variant. The culture was shaken at 37° C. and 250 rpm until the OD₆₀₀ was 0.7 (approximately 2 hours). The culture was placed on ice for 30 minutes, and isopropyl β-D-1-thiogalactopyranoside (IPTG) was added to final concentrations of 1 mM. The incubator temperature was reduced to 20.5° C., and the culture was allowed to shake for 24 hours at 180 rpm. Cells were harvested by centrifugation (4° C., 15 min, 4,000×g) and resuspended in KPi buffer (pH 7.4) and adjusted to OD₆₀₀=20. The whole-cell suspension was placed on ice and bubbled with Ar for 15 min.

### Anaerobic Techniques

Unless otherwise stated, spectroscopic samples were prepared in an MBraun UNIlab glovebox circulated under a positive pressure of N2(g). Sav HppD Az1 was rendered anoxic by vacuuming and sparging the protein (˜7 cycles) with Ar(g) in a round bottom flask connected to a Shlenk line. All buffers and compounds were prepared within the glovebox to render a uniform anaerobic environment.

### Example 2

### Non-Native Azidation by Multiple Non-Heme Metalloenzymes

This example covers the reprogramming of multiple non-heme iron enzymes to catalyze abiological C(sp³)-H azidation reactions via iron-catalyzed radical relay. These biocatalytic transformations use amidyl radicals as hydrogen atom abstractors and Fe(III)—N₃ intermediates as radical trapping agents. A high-throughput screening platform based on click chemistry was established for rapid optimization of the catalytic performance of enzymes identified. The final optimized variants function in whole Escherichia coli cells and deliver a range of azidation products with up to 10600 total turnovers and 93% enantiomeric excess. Given the high prevalence of radical relay reactions in organic synthesis and the large diversity of non-heme iron enzymes, we envision that this discovery will stimulate future development of metalloenzyme catalysts for synthetically useful transformations unexplored by natural evolution.

Azidation of the N-fluoroamide substrate N-(tert-butyl)-2-ethyl-N-fluorobenzamide (1NF):

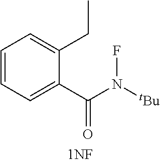 [SMILES: CCc1ccccc1C(=O)N(F)C(C)(C)C]

was tested using a panel of nine functionally diverse non-heme iron enzymes under whole-cell conditions. The reactions primarily produced the benzylic azidation product 1N, as well as small amounts of intramolecular fluorine transfer product 1F and dehalogenation product 1A:

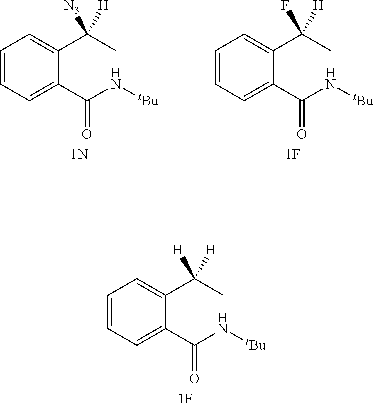

The reactions were performed by adding ferrous ammonium sulfate (10 μL, 100 mM in water), sodium azide (10 μL, 1 M in water), and N-fluoroamide substrate 1NF (10 μL, 400 mM in DME) to E. coli harboring non-heme iron enzymes (400 μL, adjusted to OD₆₀₀=40) in a 2 mL screw top GC vial. The vial was capped and shaken at 680 rpm at room temperature for 24 hours. At the end of the reaction, the vial was opened and the reaction was quenched with 0.8 mL of a hexanes/ethylacetate solution (4:6 v/v) of internal standard 1,2,3-trimethoxybenzene (0.5 mM final concentration). The reaction mixture was transferred to a 2 mL microcentrifuge tube, vortexed (10 seconds, 3 times), then centrifuged (14,000×g, 5 min) to completely separate the organic and aqueous layers. An aliquot (200-300 μL) of the organic layer was used for product quantification via GCMS and enantioselectivity via chiral HPLC or chiral GC. The results of these analyses are summarized in TABLE 2.

TABLE 2

| | | | | | | 1F | 1A |
|---|---|---|---|---|---|---|---|
| Entry | Enzyme | Organism | Uniprot No. | 1N % | e.r.^{d} | % | % |
| 1 | 4-hydroxyphenylpyruvate | Streptomyces | Q53586 | 33.0 ± 2.0 | 63:37 | 3.7 | 10.0 |
| | dioxygenase (HppD)^{a} | avermitilis | | | | | |
| 2 | Proline cis-4-hydroxylase | Mesorhizobium | Q989T9 | 0.5 | n.d. | 0.4 | 5.0 |
| | (P4H)^{b} | japonicum | | | | | |
| 3 | Iron/2-oxoglutarate- | Hapalosiphon | A0A067YX61 | 0.1 | n.d. | 0.3 | 2.5 |
| | dependent halogenase | welwitschii | | | | | |
| | WelO5^{b} | | | | | | |
| 4 | polyoxin hydroxylase PolL^{c} | Streptomyces | J7FW05 | 0.6 | n.d. | 0.4 | 8.6 |
| | | aureochromogenes | | | | | |
| 5 | Prolyl 4-hydroxylase^{c} | Bacillus | A0A4Y1WAP5 | n.d. | n.d. | n.d. | n.d. |
| | | anthracis | | | | | |
| 6 | Prolyl 4-hydroxylase^{bc} | Paramecium | Q84406 | 0.2 | n.d. | 0.7 | 6.7 |
| | | bursaria | | | | | |
| | | Chlorella virus | | | | | |
| 7 | Iron/2-oxoglutarate- | Emericella | Q5AR53 | 0.1 | n.d. | 0.5 | 5.0 |
| | dependent halogenase AsqJ^{c} | nidulans | | | | | |
| 8 | Isopenicillin N synthase | Emericella | P05326 | 4.0 ± 0.1 | 44:56 | 1.5 | 8.0 |
| | (IPNS)^{b} | nidulans | | | | | |
| 9 | (S)-2- | Pseudomonas | Q9JN69 | 0.1 | n.d. | 0.3 | 3.3 |
| | hydroxypropylphosphonic | syringae | | | | | |
| | acid epoxidase Psf4^{b} | | | | | | |

| | | | | | |
|---|---|---|---|---|---|
| 10 | KPi buffer without whole-cell catalyst | 0.1 | n.d. | 0.3 | 0.5 |
| 11 | Sav HppD H187A H270A as the catalyst | 0.2 | n.d. | 0.2 | 7.5 |
| 12 | wt Sav HppD, no azide addition | n.d. | n.d. | 2.2 | 6.5 |

^{a}1N %, 1F %, and 1A % refer to the yield of 1N, 1F, and 1A, respectively.
e.r. denotes product enantiomeric ratio.
^{b}pET-22b(+) was used as the cloning vector.
^{c}pET-28a(+) was used as the cloning vector.
^{d}not determined (n.d.)

While numerous metalloenzymes performed the azidation reaction, a (4-hydroxyphenyl)pyruvate dioxygenase from Streptomyces avermitilis (Sav HppD) provided the highest yield of 1N, including a total turnovers (TTN) of 250, an enantiomeric ratio (e.r.) of 63:37, and a chemoselectivity of 9:1 for azidation over fluorination product. Only trace amount of azidation product was obtained in a reaction lacking Sav HppD. Moreover, mutating the two iron-coordinating histidines to alanines abolished the enzyme activity while retaining the fold of wt Sav HppD, supporting the proposal that reaction occurs at the 2-His-1-carboxylate iron center. The unazidated amide product was also detected in trace amount, but was likely formed via an unidentified non-enzymatic process, as the double alanine mutant afforded this product in a yield comparable to that of the wild-type enzyme.

### Example 3

### Directed Evolution of a Non-Heme Metalloenzyme

This example covers the improvement of Sav HppD performance via directed evolution. Computational modeling was performed on the wild-type enzyme with both azide and 1NF substrate bound. Fifteen active site residues—H187, V189, N191, L228, S230, P243, N245, Q255, Q269, H270, F336, E349, F364, L367, and F368—were selected for optimization. These residues mainly reside o-helix, s barrel of the C-terminal domain, a-helix, β barrel of the C-terminal domain, and loops surrounding the active site.

A high-throughput screening (HTS) platform based on copper-catalyzed azide-alkyne cycloaddition (CuAAC) was utilized for Sav HppD variants, and provided reliable quantification of enzymatic azidation products with a coefficient of variation of 9% and a detection limit of 4 μM. With this HTS platform, more than 5,000 clones generated through error-prone PCR or site-saturation mutagenesis were evaluated. Results of 1NF azidation with select variants are summarized in TABLE 3.

TABLE 3

| Entry | Acronym | Mutations from wt Sav HppD | TTN | e.r. | 1N/1F | 1N/1A |
|---|---|---|---|---|---|---|
| 1 | wt Say HppD | None | 250 ± 10 | 63:37 | 9.0 | 3.0 |
| 2 | HppD AQ | V189A N245Q | 320 ± 20 | 76:24 | 18.8 | 7.8 |
| 3 | HppD AQI | V189A N245Q L367I | 410 ± 10 | 79:21 | 27.6 | 9.8 |
| 4 | HppD AQAI | V189A N245Q Q255A L367I | 760 ± 20 | 87:13 | 35.5 | 9.3 |
| 5 | HppD Az1 | V189A F216A N245Q Q255A | 1340 ± 40 | 86:14 | 53.8 | 15.5 |
| | | P243A L367I | | | | |
| 6 | HppD ALGFPI | V189A S230L P243G N245F | 430 ± 30 | 94:6 | 33.2 | 13.4 |
| | | Q255P L367I | | | | |
| 7 | HppD Az2 | V189A N191A S230L P243G | 490 ± 20 | 96:4 | 30.6 | 19.3 |
| | | N245F Q255P L367I | | | | |

TTN denotes total turnover;
e.r. denotes enantiomer ratio;
1N/1F denotes the ratio of 1N to 1F among products.
1N/1A denotes the ratio of 1N to 1A among products

A sextuple mutant Sav HppD V189A F216A P243A N245Q Q255A L367I (denoted as Sav HppD Az1) furnished the product with 1340 TTN and 87:13 e.r. This evolution campaign, did not identify an enzyme variant with an e.r. higher than 87:13. This result indicates that mutations that were beneficial for improving activity might not necessarily lead to an increase in enantioselectivity, which might be due to the differences in substrate positioning and geometric requirement for the rate-determining N-F activation step and the enantio-determining azide rebound step as revealed by molecular dynamics simulation. Some of the libraries were then reevaluated with chiral HPLC and additional rounds of evolution aided by computational modelling. A septuple mutant Sav HppD V189A N191A S230L P243G N245F Q255P L367I (denoted as Sav HppD Az2) showed an enantioselectivity of 96:4 e.r. and 490 TTN.

Kinetic analyses for wild-type Sav HppD, Az1, and Az2 mediated 1NF azidation were performed in an anaerobic chamber. Ferrous ammonium sulfate (10 μL, 100 mM in water) and sodium azide (10 μL, 1 M in water) were added to a buffer solution containing purified Sav HppD protein variant (20 M, 2.4 mL) and the solution was shaken at 600 rpm for 5 minutes. A 1,2-dimethoxyethane solution of N-fluoroamide substrate 1NF was added to the solution (final concentration ranging from 0.25 mM to 15 mM in reaction solution). An aliquot of 100 μL of the reaction mixture was removed at 3, 6, 9, 12, and 15 minutes and quenched by vortexing with 300 μL 6:4 EtOAc/hexanes solution containing 0.5 mM (final concentration) internal standard 1,2,3-trimethoxybenzene. After centrifugation at 12,000 rpm for 10 mins, an aliquot (200 μL) of the organic layer was taken for GCMS analysis for product quantification. Experiments were performed in triplicates, and are summarized in FIG. 10. The Az1 variant exhibited a 4.1-fold increase in k_{cat} and a 1.7-fold increase in K_{M} over the wild-type enzyme (29.4 min⁻¹ (Az1) vs 7.20 min−1 (wt) for k_{cat} and 790 μM (Az1) vs 470 μM (wt) for K_{M}), whereas the more enantioselective Az2 variant displayed a 9-fold decrease in k_{cat} (3.39 min⁻¹) and a 6.6-fold decrease in K_{M} (120 μM). Overall, both variants showed around 2-fold improvement in catalytic efficiency (k_{cat}/K_{M}) compared to that of the wild-type enzyme.

### Example 4

### Azidation Reaction Condition Optimization

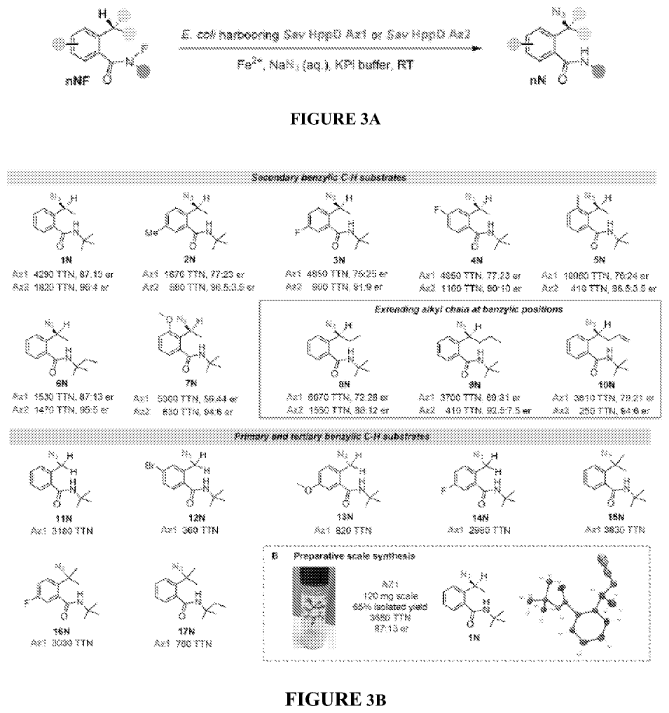

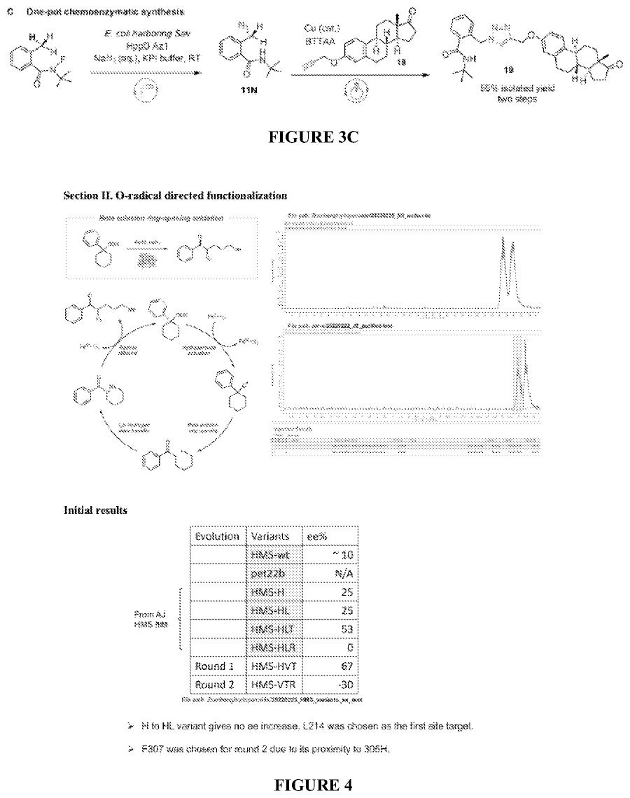

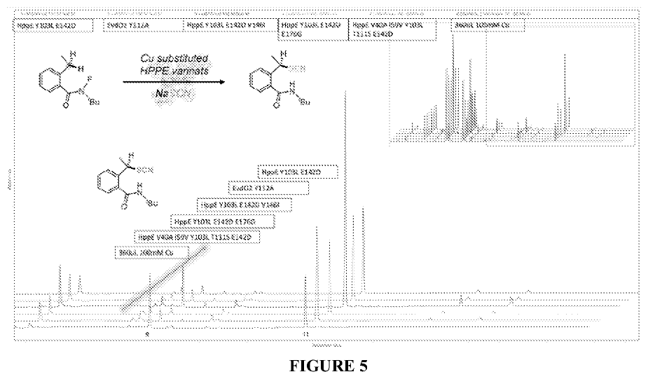

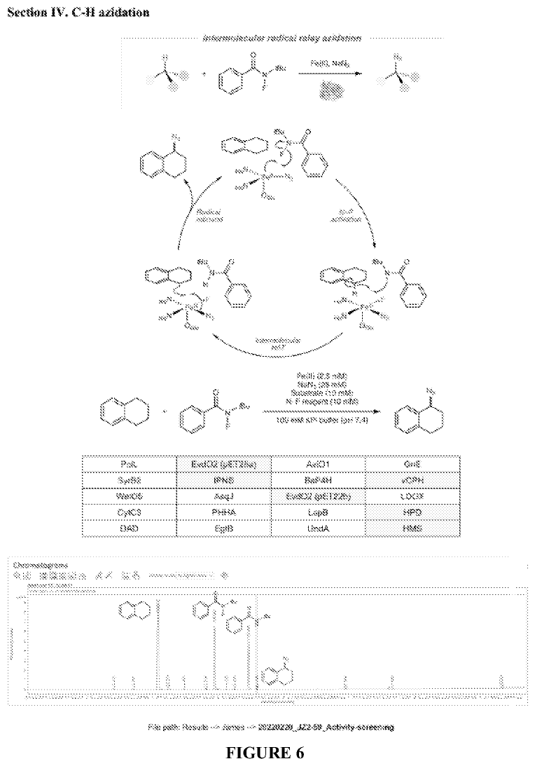

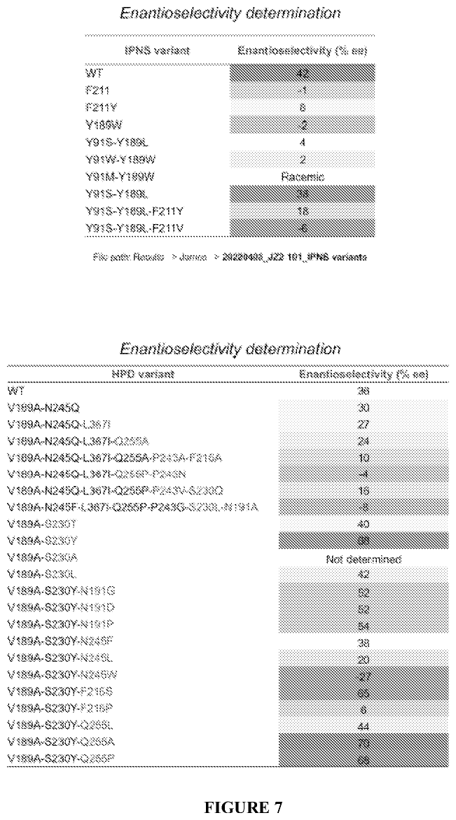

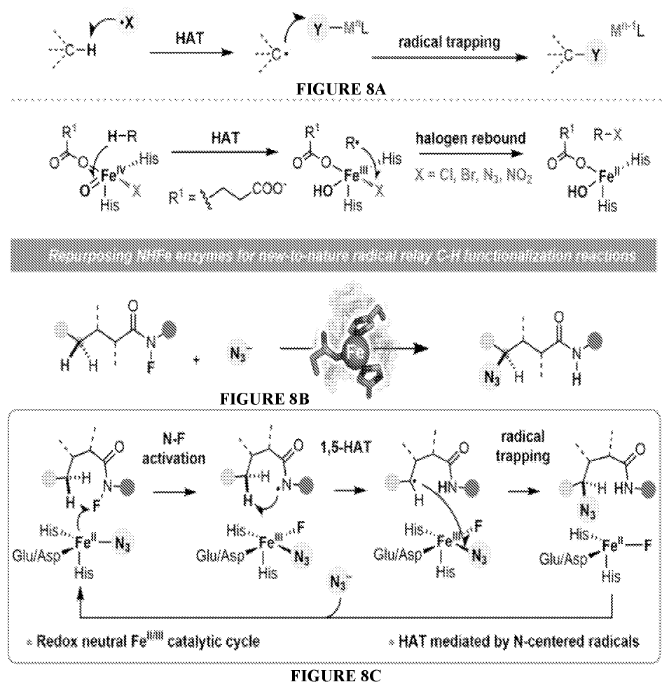

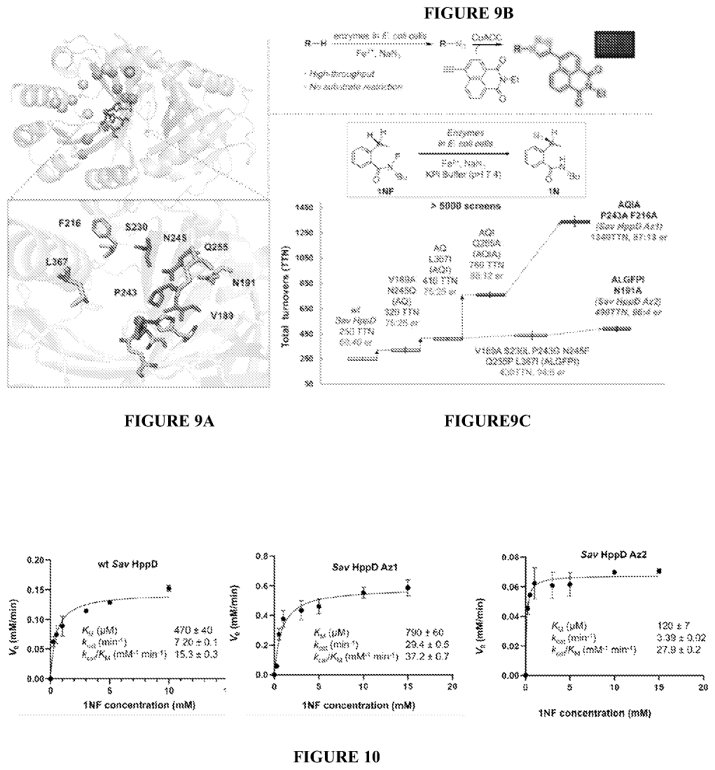

This example covers optimization of reaction conditions and analysis of multiple N-fluoroamide substrates with the sextuple and septuple Sav HppD variants Az1 and Az2 from Example 2. A scheme for this reaction is shown in FIG. 3A.

Reaction condition optimization was performed in an anaerobic chamber. Ferrous ammonium sulfate (10 μL, 100 mM in water), sodium azide (10 μL, 1 M in water), and N-fluoroamide substrate 1NF (10 μL, 400 mM in DME) were added to E. coli harboring non-heme iron enzymes (400 μL, adjusted to OD₆₀₀=10) in a 2 mL screw top GC vial. The vial was capped and shaken at 680 rpm at room temperature for 24 hours. At the end of the reaction, the vial was opened and the reaction was quenched with 0.8 mL of a hexanes/ethylacetate solution (4:6 v/v) of internal standard 1,2,3-trimethoxybenzene (0.5 mM final concentration). The reaction mixture was transferred to a 2 mL microcentrifuge tube, vortexed (10 seconds, 3 times), then centrifuged (14,000×g, 5 min) to completely separate the organic and aqueous layers. An aliquot (200-300 μL) of the organic layer was used for product quantification via GCMS and enantioselectivity via chiral HPLC or chiral GC. Protein concentrations in whole cell solutions were determined using cell lysis and protein concentration measurement. Exemplary condition optimization results with Sav HppD Az1 are shown in TABLE 4.

TABLE 4

| Variations from initial conditions | TTN | e.r. | 1/1F | 1/1A |
|---|---|---|---|---|
| Az1 variant | 1340 ± 40 | 87:13 | 54.0 | 15.5 |
| NaN₃ (20 μL, 1M), Az1 variant | 1550 ± 40 | 87:13 | 52.0 | 14.0 |
| OD₆₀₀ = 10, Az1 variant | 1940 ± 20 | 87:13 | 48.0 | 16.4 |
| 1NF (10 μL, 800 mM), OD₆₀₀ = 10, Az1 variant | 2480 ± 30 | 87:13 | 52.0 | 18.0 |
| 1NF (10 μL, 1.5M), Fe²⁺ (20 μL, 100 mM) | 3100 ± 60 | 87:13 | 47.0 | 19.5 |
| NaN₃ (40 μL, 1M), OD₆₀₀ = 10, Az1 variant | | | | |
| INF (20 μL, 1.5M), Fe²⁺ (20 μL, 100 mM) | 4290 ± 50 | 87:13 | 55.0 | 20.0 |
| NaN₃ (40 μL, 1M), OD₆₀₀ = 10, Az1 variant | | | | |
| 1NF (20 μL, 1.5M), Fe²⁺ (20 μL, 100 mM) | 260 ± 30 | 96:4 | 18.0 | 7.0 |
| NaN₃ (40 μL, 1M), OD₆₀₀ = 10, Az2 variant | | | | |
| 1NF (10 μL, 400 mM), Fe²⁺ (10 μL, 100 mM) | 490 ± 20 | 96:4 | 30.6 | 19.3 |
| NaN₃ (10 μL, 1M), OD₆₀₀ = 10, Az2 variant | | | | |
| 1NF (20 μL, 1.5M), Fe²⁺ (20 μL, 100 mM) | 1820 ± 50 | 96:4 | 34.0 | 21.0 |
| NaN₃ (40 μL, 1M), OD₆₀₀ = 40, Az2 variant | | | | |

TTN denotes total turnover; e.r. denotes enantiomer ratio; 1N/1F denotes the ratio of 1N to 1F among products. 1N/1A denotes the ratio of 1N to 1A among products

Across the substrates and conditions tested, Sav HppD Az1 generally exhibited higher activity but lower enantioselectivity than Sav HppD Az2. The enzymatic reaction tolerates a range of aromatic substitution patterns with total turnovers up to 10060 and enantiomeric ratio up to 96.5:3.5 (product 5N, FIG. 3B). Substrates with an extended alkyl chain at the benzylic position were well tolerated, providing products in moderate-to-good TTNs and enantioselectivity (products 8N-10N, FIG. 3B). The amide nitrogen substituent also impacts enzyme performance, as evidenced by a decrease in activity when a larger N-tert-amyl group is substituted for the N-tert-butyl group (1N and 6N, 15N and 17N, FIG. 3B).

We also tried to extend the scope of N-radical precursors and replace azide with other halide or pseudohalide anions, the results of which analyses are summarized in TABLE 5. For these analyses. ferrous ammonium sulfate (10 μL, 100 mM in water), sodium halide or pseudohalide solution (10 μL, 1 M in water), and N-fluoroamide substrate 1NF (10 μL, 400 mM in DME) were added to a 2 mL vial containing Sav HppD Az1 cell lysate (400 μL, obtained from OD₆₀₀=20 cell suspension) in an anerobic chamber. The vial was capped and shaken at 680 rpm at room temperature for 24 hours. At the end of the reaction, the vial was opened and the reaction was quenched with 0.8 mL of a hexanes/ethylacetate solution (4:6 v/v) of internal standard 1,2,3-trimethoxybenzene (0.5 mM final concentration). The reaction mixture was transferred to a 2 mL microcentrifuge tube, vortexed (10 seconds, 3 times), then centrifuged (14,000×g, 5 min) to completely separate the organic and aqueous layers. An aliquot (200-300 μL) of the organic layer was used for product quantification via GCMS. 1X/internal refers to the ratio of peak area of 1X over that of the internal standard as determined by GCMS total ion chromatogram. 1F/internal and 1A/internal were defined and calculated accordingly.

TABLE 5

| Nucleophile Source | IF/internal | IA/internal |
|---|---|---|
| NaF | 0.04 | 2.5 |
| NaBr | 0.04 | 2.4 |
| NaOCN | 0.03 | 2.1 |
| NaSCN | 0.05 | 2.7 |
| NaNO₂ | 0.03 | 1.9 |
| NaCl | 0.04 | 2.3 |
| NaCN | 0.04 | 2.4 |

As suggested by Mössbauer studies, the inability of our method to incorporate other anionic ligands might be due to a much weaker binding of these anions to the Fe(II) center of the enzymes. In a larger scale reaction, Sav HppD Az1 furnished 1N in 65% isolated yield at 120 mg scale with undiminished enantioselectivity (FIG. 3B). Single crystals of 1N were analyzed to assign its absolute configuration as S by X-ray crystallography. The primary organic azide 11N was also produced at preparative scale and subsequently converted it into an estrone derivative 18 via a CuAAC reaction (FIG. 3C). This chemoenzymatic two-step synthesis yielded the triazole product 19 in 55% isolated yield, demonstrating the potential of this platform to produce highly functionalized molecules when used in tandem with biocompatible reactions.

### Example 5

### Mechanistic Studies of Non-Heme Metalloenzyme Mediated Azidation

Mechanistic studies were performed on Sav HppD to determine its azidation mechanism. Addition of N₃⁻ to Sav HppD Az1·Fe(III) complex induced the formation of two quadrupole doublets in Mössbauer spectrum with isomer shifts (δ) of 1.20 and 1.17 mm/s and quadrupole splittings (ΔE_{Q}) of 2.29 and 2.97 mm/s, respectively. The observation of two quadrupole doublets may reflect different azide binding configurations to the Fe(II) center. Electron paramagnetic resonance (EPR) measurements were then performed on nitric oxide (NO)-bound Sav HppD Az1·Fe(II) complex whose prominent g˜4 EPR resonance was used to monitor the interactions between the substrate and the non-heme iron center. Adding azide to Sav HppD Az1·Fe(II)·NO complex increased the rhombicity (E/D) of the g˜4 signal from 0.014 to ˜0.017, the further addition of 1NF continued increasing the signal rhombicity (E/D=0.023).

These observations suggest that both N₃ and 1NF interact with the Fe(II) center of Sav HppD Az1. To demonstrate an Fe(III)—N₃ species is involved in the reaction, Sav HppD Az1·Fe(II)—N₃ was incubated with an N-fluoroamide 18NF that lacked the reactive benzylic C—H bonds. A slow accumulation of a red species was observed with an optical absorption centered at 505 nm, which likely originated from the Fe(III)—N₃ ligand-to-metal charge transfer band (20-22). The EPR signal of this red species was located at g˜4.3, further confirming its oxidation state was high spin (S=5/2) Fe(III) (see section X of the SI). In this study, the formation of a minor stable organic radical centered at g=2 was also observed. Although further studies are needed to characterize this radical species, it was speculated to be a secondary radical formed via the quench of the initial amidyl radical, as this g=2 signal was not observed when incubating Sav HppD Az1·Fe(II)·N₃ with the model N-fluoraoamide 1NF.

### Example 6

### Computational Modeling of Azidation within a Non-Heme Metalloenzyme Active Site

Computational modelling was performed on wild-type and variant Sav HppD to understand the molecular basis of the azidation reaction, and to identify mutations which can enhance efficiency, turnover, enantioselectivity, and chemoselectivity for this reaction. Focusing on enantioselective variant Sav HppD Az2, MD simulations showed that V189A and P243G generated more space to accommodate iron-bound azide in the active site, indicating that increasing active site volume can promote azidation. In wt Sav HppD, N191, N245 and S230 participated in a hydrogen bonding network with Q269 for native substrate positioning. Introducing the mutations N191A, S230L, and P243G disrupted this network. These mutations together with N245F and L367I created a hydrophobic environment to accommodate N-fluoroamide substrates for N-F activation and position the ethyl group of the substrate closer to the iron-bound azide in a restricted and preorganized conformation for the subsequent reaction steps.

### Example 7

### Azidation Enantioselectivity of Multiple Non-Heme Metalloenzyme Variants

This example covers C—H bond functionalization of a benzylic carbon by a non-heme metalloenzyme. The reaction utilized the organic substrate 1,2,3,4-tetrahydronaphthalene, the radical precursor N-(tert-butyl)-N-fluorobenzamide, and the nucleophile source NaN₃ as overviewed in SCHEME 5. Multiple Sav HppD variants were tested, and exhibited enantioselectivity of between −27% and 68%. The results of these analyses are summarized in FIG. 7.

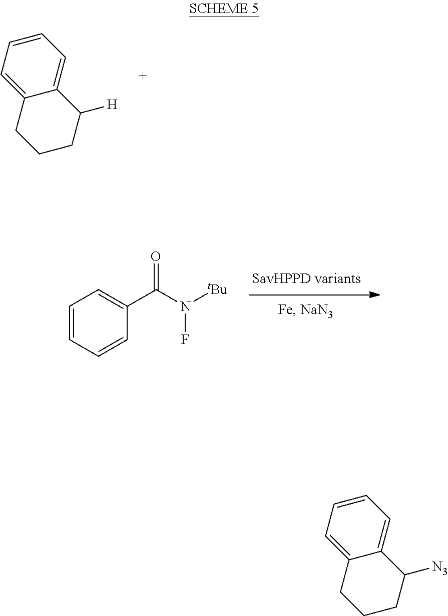 [SMILES: c1ccc2c(c1)CCCC2 ; CC(C)(C)N(F)C(=O)c1ccccc1 ; [N-]=[N+]=NC1CCCc2ccccc21]

### Example 8

### Non-Heme Metalloenzyme-Mediated Benzylic Addition

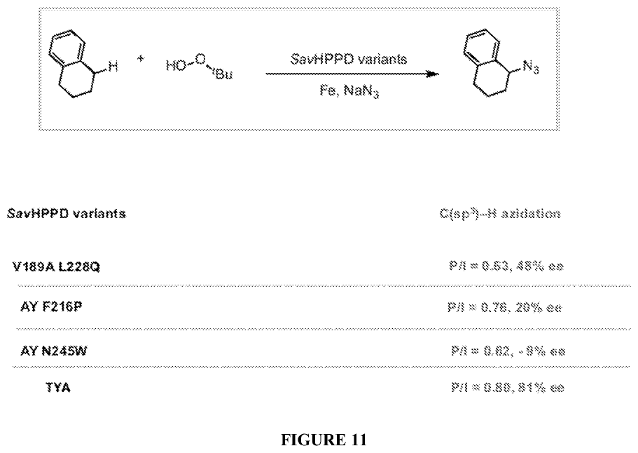

This example covers C—H bond functionalization of a benzylic carbon by a non-heme metalloenzyme. The reaction utilized the organic substrate 1,2,3,4-tetrahydronaphthalene, the radical precursor (tert-butyl)-hydroperoxide, and the nucleophile source NaN₃ as overviewed in SCHEME 6. Multiple Sav HppD variants were tested, and exhibited enantioselectivity of between −9% and 81%. The results of these analyses are summarized in FIG. 11.

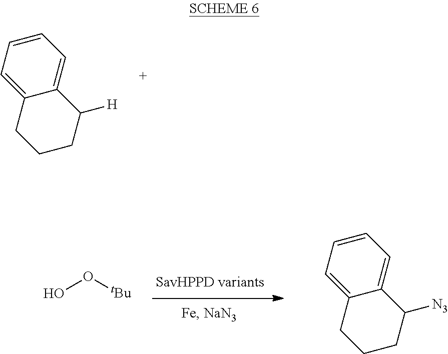 [SMILES: c1ccc2c(c1)CCCC2 ; CC(C)(C)OO ; [N-]=[N+]=NC1CCCc2ccccc21]

Although the invention has been described with reference to the above examples, it will be understood that modifications and variations are encompassed within the spirit and scope of the invention. Accordingly, the invention is limited only by the following claims.

1. A non-heme metalloenzyme comprising (i) at least about 70% sequence identity to SEQ ID NO:1, and comprising at least 1 mutation relative to SEQ ID NO:1, or (ii) at least at least 98.2%, at least 98.4%, at least 98.6%, at least 98.8%, at least 99%, at least 99.2%, at least 99.4%, or at least 99.6% sequence identity to SEQ ID NO:2 or SEQ ID NO:3.

2-3. (canceled)

4. The non-heme metalloenzyme of claim 1, wherein the non-heme metalloenzyme comprises at least 1 mutation at SEQ ID NO:1 position H187, V189, N191, L228, S230, P243, N245, Q255, Q269, H270, F336, E349, F364, L367, F368, or a combination thereof, optionally wherein the mutations are V189A, N191A, F216A, S230L, P243A, P243G, N245Q, N245F, Q255A, Q255P, and L367I.

5-6. (canceled)

7. The non-heme metalloenzyme of claim 1, wherein the at least 1 mutation diminishes active site volume in the non-heme metalloenzyme.

8. (canceled)

9. A composition comprising a non-heme metalloenzyme of claim 1, an organic substrate comprising a C—H bond, and one or more of a halogen source, a nucleophile source, and a radical precursor.

10. A method for modifying an organic substrate comprising:
contacting the organic substrate with a non-heme metalloenzyme;
abstracting a hydrogen atom from the organic substrate; and
coupling a nucleophile to the organic substrate,
thereby converting the organic substrate to a modified organic substrate.

11. The method of claim 10, wherein the non-heme metalloenzyme comprises an iron cofactor, a copper cofactor, a cobalt cofactor, a manganese cofactor, a nickel cofactor, or a chromium cofactor.

12. (canceled)

13. The method of claim 12, wherein the iron cofactor has a +2 oxidation state, interconverts between +2 and +3 oxidation states and/or does not adopt a +4 oxidation state.

14-15. (canceled)

16. The method of claim 10, wherein the non-heme metalloenzyme comprises at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to any one of SEQ ID NO:1-16.

17. (canceled)

18. The method of claim 16, wherein the non-heme metalloenzyme comprises at least one, at least two, at least three, at least four, at least five, at least six, at least seven, at least eight, at least nine, at least ten, at least eleven, at least twelve, at least thirteen, at least fourteen, or fifteen mutations relative to SEQ ID NO:1 at positions selected from H187, V189, N191, L228, S230, P243, N245, Q255, Q269, H270, F336, E349, F364, L367, and F368.

19. The method of claim 10, wherein the non-heme metalloenzyme catalyzes the coupling between the nucleophile and the organic substrate.

20. The method of claim 10, wherein the nucleophile is (i) bonded to a metal cofactor of the non-heme metalloenzyme prior to the coupling or (ii) coupled to the carbon atom from which the hydrogen atom is abstracted.

21. The method of claim 10, wherein the hydrogen atom is abstracted (i) from a carbon atom of the organic substrate or (ii) by an organic radical generated by the non-heme metalloenzyme.

22. (canceled)

23. The method of claim 10, wherein the nucleophile is an azide, a halogen, a nitrile, a thiocyanate, a nitro, a cyanide, an alkoxide, a thiolate, an amine, a sulfonamide, an amide, a heteroaryl, or a trifluoromethyl.

24. (canceled)

25. The method of claim 23, wherein the method has a chemoselectivity for azidation over fluorination of greater than about 3:2, greater than about 2:1, greater than about 3:1, greater than about 4:1, greater than about 5:1, greater than about 6:1, greater than about 7:1, greater than about 8:1, greater than about 9:1, greater than about 10:1, greater than about 12:1, greater than about 15:1, greater than about 20:1, or greater than about 25:1.

26. The method of claim 10, wherein the nucleophile is derived from a nucleophile source with a structure according to any one of Formulas (VIII)—(XVII) or (XIX): 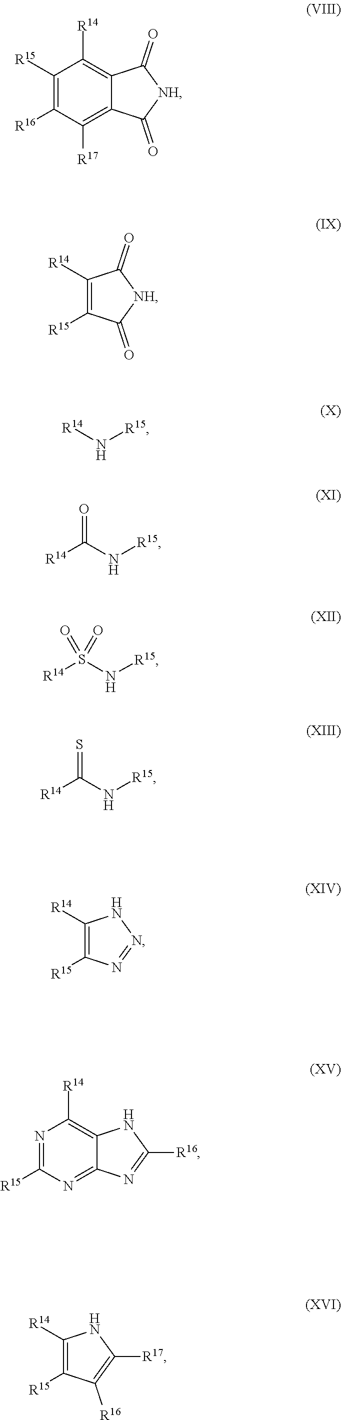
or M⁺X⁻ (XIX);
wherein each instance of R¹⁴, R¹⁵, R¹⁶ and R¹⁷ is independently —H, optionally substituted C₁₋₁₈ alkyl, C₁₋₁₈ polyfluoroalkyl, optionally substituted C₂₋₁₈ alkenyl, optionally substituted C₂₋₁₈ alkynyl, optionally substituted C₆₋₁₀ aryl, optionally substituted 6- to 10-membered heteroaryl, optionally substituted 6- to 10-membered heterocyclyl, cyano, halo, nitro, —NR¹⁸R¹⁹, —BR²¹R²², —SiR¹⁸R¹⁹R²⁰, —C(O)OR¹⁸, —C(O)SR¹⁸, —C(O)NR¹⁸R¹⁹, —C(O)R¹⁸, —C(O)ONR¹⁸R¹⁹, —C(O)NR¹⁸OR¹⁹, —C(O)C(O)OR¹⁸, —S(O)OR¹⁸, —S(O)SR¹⁸, —S(O)NR¹⁸R¹⁹, —S(O)R¹⁸, —S(O)ONR¹⁸R¹⁹, —S(O)NR¹⁸OR¹⁹, —S(O)C(O)OR¹⁸, —S(O)₂OR¹⁸, —S(O)₂SR¹⁸, —S(O)₂NR¹⁸R¹⁹, —S(O)₂R¹⁸, —S(O)₂ONR¹⁸R¹⁹, —S(O)₂NR¹⁸OR¹⁹, —S(O)₂C(O)OR¹⁸, or —P(O)(OR¹⁸)(OR¹⁹) each instance of R¹⁸, R¹⁹, and R²⁰ is independently —H, C₁-C₃ alkyl, or C₁-C₃ haloalkyl;
each instance of R²¹ and R²² is independently —H, C₁-C₃ alkyl, C₁-C₃ haloalkyl, or —OR¹⁸;
M⁺ is Na⁺, K⁺, Cs⁺, or [N(R¹²)₄]⁺;
X⁻ is F⁻, Cl⁻, Br⁻, I⁻, N₃⁻, SCN⁻, CN⁻, NCO⁻, [SR¹³]⁻, or [OR¹³]⁻;
each instance of R¹² is independently —H, C₁-C₆ alkyl, or C₁-C₆ haloalkyl, or wherein two instances of R¹² are taken together along with the nitrogen to which they are attached to form a C₂-C₈ heterocycloalkyl; and
each instance of R¹³ is independently —H, C₁-C₆ alkyl, or C₁-C₆ haloalkyl.

27. (canceled)

28. The method of claim 27, wherein the organic radical is generated through homolysis of a bond on a radical precursor.

29. (canceled)

30. The method of claim 28, wherein the bond on the radical precursor is a halogen-halogen bond, a carbon-halogen bond, a nitrogen-halogen bond, or an oxygen-oxygen bond.

31. The method of claim 28, wherein the radical precursor is coupled to the organic substrate and has a structure according to any one of Formulas (I)—(VII): 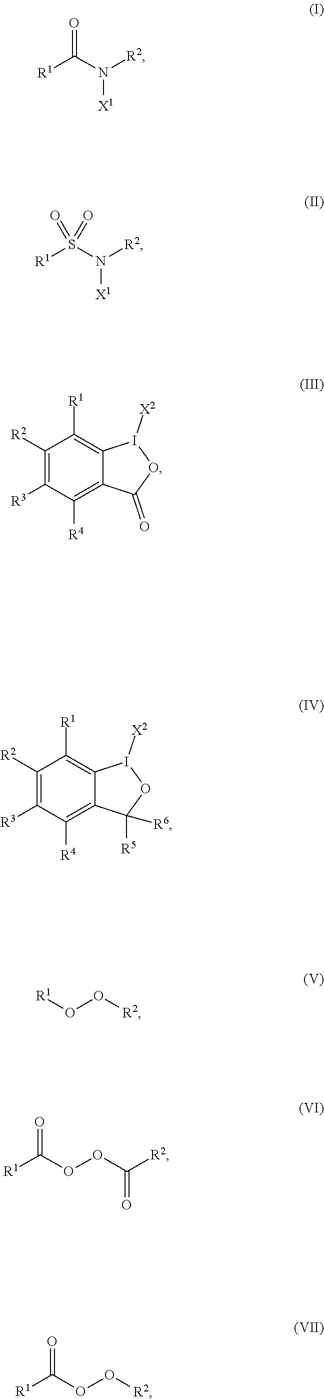
wherein each instance of R¹, R², R³, R⁴, R⁵, and R⁶ is independently the organic substrate, —H, optionally substituted C₁₋₁₈ alkyl, optionally substituted C₁₋₁₈ polyfluoroalkyl, optionally substituted C₂₋₁₈ alkenyl, optionally substituted C₂₋₁₈ alkynyl, optionally substituted C₆₋₁₀ aryl, optionally substituted 6- to 10-membered heteroaryl, optionally substituted 6- to 10-membered heterocyclyl, cyano, halo, nitro, —NR⁷R⁸, —BR¹⁰R¹¹, —SiR⁷R⁸R⁹, —C(O)OR⁷, —C(O)SR⁷, —C(O)NR⁷R⁸, —C(O)R⁷, —C(O)ONR⁷R⁸, —C(O)NR⁷OR⁸, —C(O)C(O)OR⁷, —S(O)OR⁷, —S(O)SR⁷, —S(O)NR⁷R⁸, —S(O)R⁷, —S(O)ONR⁷R⁸, —S(O)NR⁷OR⁸, —S(O)C(O)OR⁷, —S(O)₂OR⁷, —S(O)₂SR⁷, —S(O)₂NR⁷R⁸, —S(O)₂R⁷, —S(O)₂ONR⁷R⁸, —S(O)₂NR⁷OR⁸, —S(O)₂C(O)OR⁷, or —P(O)(OR⁷)(OR⁸);
each instance of R⁷, R⁸, and R⁹ is independently —H, C₁-C₃ alkyl, or C₁-C₃ haloalkyl;
each instance of R¹⁰ and R¹¹ is independently —H, C₁-C₃ alkyl, C₁-C₃ haloalkyl, or —OR⁷;
each instance of X¹ is independently —F, —Cl, —Br, or —I; and
each instance of X² is independently —F, —Cl, or —Br.

32. The method of claim 10, wherein the modified organic substrate is coupled to the nucleophile through a carbon-nitrogen bond, a carbon-sulfur bond, a carbon-carbon bond, or a carbon halogen bond.

33. The method of claim 10, wherein the organic substrate contains a carbon-halogen or nitrogen-halogen bond that is not cleaved during the method.

34. The method of claim 10, further comprising dehalogenating the organic substrate.

35. The method of claim 10, wherein the method is performed under anaerobic conditions and/or in the presence of a cell that expresses the non-heme metalloenzyme.

36. The method of claim 10, wherein the modified organic substrate has an enantiomeric ratio of at least about 60:40, at least about 65:35, at least about 70:30, at least about 75:25, at least about 80:20, at least about 85:15, at least about 90:10, or at least about 95:5.

37-38. (canceled)

39. The method of claim 10, wherein the organic substrate has a structure according to Formula (XVIII): 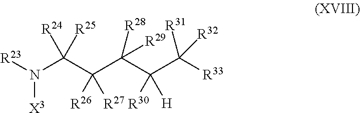
wherein R²³, R²⁴, R²⁵, R²⁶, R²⁷, R²⁸, R²⁹, R³⁰, R³¹, R³², and R³³ are independently —H, optionally substituted C₁₋₁₈ alkyl, C₁₋₁₈ polyfluoroalkyl, optionally substituted C₂₋₁₈ alkenyl, optionally substituted C₂₋₁₈ alkynyl, optionally substituted C₆₋₁₀ aryl, optionally substituted 6-10-membered heteroaryl, optionally substituted 6- to 10-membered heterocyclyl, cyano, halo, nitro, —NR³⁴R³⁵, —BR³⁷R³⁸, —SiR³⁴R³⁵R³⁶, —C(O)OR³⁴, —C(O)SR³⁴, —C(O)NR³⁴R³⁵, —C(O)R³⁴, —C(O)ONR³⁴R³⁵, —C(O)NR³⁴OR³⁵, —C(O)C(O)OR³⁴, —S(O)OR³⁴, —S(O)SR³⁴, —S(O)NR³⁴R³⁵, —S(O)R³⁴, —S(O)ONR³⁴R³⁵, —S(O)NR³⁴OR³⁵, —S(O)C(O)OR³⁴, —S(O)₂OR³⁴, —S(O)₂SR³⁴, —S(O)₂NR³⁴R³⁵, S(O)₂R³⁴, S(O)₂ONR³⁴R³⁵, —S(O)₂NR³⁴OR³⁵, —S(O)₂C(O)OR³⁴, or —P(O)(OR³⁴)(OR³⁵);
each instance of R³⁴, R³⁵, and R³⁶ is independently —H, C₁-C₃ alkyl, or C₁-C₃ haloalkyl;
each instance of R³ and R³⁸ is independently —H, C₁-C₃ alkyl, C₁-C₃ haloalkyl, or —OR³⁴;
X₃ is —F, —Cl, —Br, or —I, and
X³ is abstracted by the non-heme metalloenzyme.

40. (canceled)

41. A method of functionalizing C(sp³)-H bonds comprising:
using reprogramed metalloenzymes to perform radical-relay C(sp³)-H functionalization;
activating a (sp³)-H bond via a reactive radical (X·) via hydrogen atom transfer (HAT);
intercepting of the resulting carbon-centered radical by a redox-reactive metal complex; and
obtaining a functionalized C—Y bond, thereby functionalizing C(sp3)-H bonds.

42. The method of claim 41, wherein the reprogrammed metalloenzymes are non-heme iron enzymes or enantioselective variants.

43. (canceled)

44. The method of claim 41, wherein the reactive radical (X·) is a nitrogen radical (N·) and/or an oxygen radical (O·).

45. The method of claim 41, wherein the functionalized C—Y bond is a C—C, C—S, C—N, C—F, and/or, C-halogen bond.
